(19) United States
(12) Patent Application Publication
(10) Pub. No.: US 2008/0194620 A1
(43) Pub. Date: Aug. 14, 2008

(54) Use of Benzodiazepine Receptor Ligands For Combating Signs of Ageing

(75) Inventors: Isabelle Besne, Puteaux (FR); Maria Dalko, Gif Sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/722,322

(22) PCT Filed: Dec. 20, 2005
(86) PCT No.: PCT/EP2005/003199

§ 371 Date: Oct. 18, 2007

Related U.S. Application Data
(60) Provisional application No. 60/641,425, filed on Jan. 6, 2005.

(30) Foreign Application Priority Data
Dec. 20, 2004 (FR) 0453083

(51) Int. Cl. A61K 31/472; A61Q 19/08; A61Q 19/00

(52) U.S. Cl. 514/307

(57) ABSTRACT

The invention relates to the cosmetic use of at least one benzodiazepine peripheral receptor antagonist in a composition containing a physiologically acceptable medium, as an agent for reducing or preventing cutaneous signs of ageing.

The present invention relates to the use of ligands of the peripheral benzodiazepine receptors (PBRs), in particular as antagonist of the peripheral benzodiazepine receptors in compositions for use in the field of the treatment or prevention of the signs of ageing of the skin, of the mucosae and/or of the integumentary appendages. The invention thus relates to the use of said PBR ligands for combating wrinkles and lines and/or loss of splendor of the skin.

Women, and even men, have a tendency to wish to appear young for as long as possible and consequently try to tone down the signs of ageing of the skin, which are notably reflected in wrinkles and lines, thinning of the epidermis and/or an appearance of skin that is flabby and withered. In this connection, advertising and the fashion industry report products that are intended to maintain, for as long as possible, skin that is radiant and wrinkle-free, which are the signs of young skin, especially as physical appearance influences one's state of mind and/or confidence.

Human skin is made up of two compartments, namely a surface compartment, the epidermis, and a deep compartment, the dermis.

The natural human epidermis is composed principally of three types of cells—the keratinocytes, which are very much in the majority, the melanocytes and the Langerhans cells.

The cells of each type contribute by their individual functions to the essential role that the skin plays in the body, notably the role of protecting the body against aggression from outside, called its “barrier function”.

The epidermis is conventionally divided into a basal layer of keratinocytes constituting the germinative layer of the epidermis, a spinous layer composed of several layers of polyhedral cells arranged on the germinative layers, one to three layers called granular layers composed of flattened cells containing distinct cytoplasmic inclusions, the keratohyaline granules and finally the horny layer (or stratum corneum), composed of an assemblage of layers of keratinocytes in the terminal stage of their differentiation, called corneocytes. The corneocytes are non-nucleated cells consisting principally of a fibrous material containing cytokeratins, surrounded by a horny envelope.

The dermis provides the epidermis with a solid support. It also supplies it with nutrients. It is principally composed of fibroblasts and an extracellular matrix, the latter being composed principally of collagen, elastin and a substance called the ground substance, components that are synthesized by the fibroblasts. It also contains leukocytes, mastocytes and perhaps tissue macrophages. Blood vessels and nerve fibers also pass through it.

The extracellular matrix of the dermis, like that of all of the body's connective tissues, is composed of proteins belonging to several broad classes: collagens, matrix glycoproteins other than the collagens (fibronectin, laminin), elastin and proteoglycans. It also contains free glycosaminoglycans (i.e. not bound to a protein).

It is now well established that specific interactions exist between these various classes of proteins to give rise to a functional tissue.

The proteoglycans (designated by the abbreviation PG) are complex macromolecules comprising a branched central protein stem, or protein network, to which numerous polysaccharide side chains called glycosaminoglycans (abbreviated to GAG) are attached.

The principal GAGs are hyaluronic acid or hyaluronan (HA), heparan sulfate (HS), heparin (HP), chondroitin, chondroitin sulfate (CS), chondroitin 4-sulfate or chondroitin sulfate A (CSA), chondroitin 6-sulfate or chondroitin sulfate C(CSC), dermatan sulfate or chondroitin sulfate B (CSB) and keratan sulfate (KS) which differs from the other glycosaminoglycans by the presence of galactose in place of uronic acid.

When associated with a protein in the form of PG, the GAGs are connected by anchoring structures to the various polypeptide chains, called core protein or carrier protein, and thus form molecules of PGs.

The GAGs can also be present in the extracellular matrix in free form, i.e. not bound to a matrix protein: this applies notably to hyaluronic acid.

During the synthesis of the PGs, the GAGs are polymerized starting from these anchoring structures.

During chronological and/or radiation-induced ageing, the dermis and the epidermis undergo many changes and degradations which, with age, are reflected in flabbiness and a loss of suppleness of the skin.

Among the components that are degraded (notably collagen and elastin), the PGs and the GAGs also undergo changes. In fact, during ageing, the fibroblasts and the keratinocytes produce less and less PGs and GAGs and their synthesis is imperfect. As a result there is considerable disorganization: the deposition of GAGs on the protein framework forming the PG is abnormal, with the result that these PGs have a reduced affinity for water and therefore there is a decrease in hydration and tonicity of the tissues.

Restoration of normal production of PGs and GAGs by the fibroblasts and keratinocytes makes some contribution to compensation of the loss of hydration of the skin.

The degradation of these matrixes therefore contributes to the phenomenon of drying and loss of suppleness of the skin.

Moreover, new keratinocytes are constantly being produced in the epidermis to make up for the continuous loss of epidermal cells from the horny layer. However, during ageing, epidermal proliferation and differentiation may be disturbed physiologically, and we may observe a tendency to a loss of balance between these two mechanisms, leading to a thinning of the living layers of the epidermis and a thickening of the horny layer.

Laugh-lines in fact result from mechanisms that are different from those that produce wrinkles due to ageing, and can appear early, from the age of 30 years.

They are actually produced as a result of the stresses exerted on the skin by the cutaneous muscles used when making facial expressions. Depending on the shape of the face, the frequency of making facial expressions and any tics, they may even appear in childhood. Age, and certain environmental factors such as exposure to the sun, are not involved in their genesis, but may deepen them further and make them permanent.

Laugh-lines are characterized by the presence of furrows around the orifices constituting the nose (nasolabial folds), the mouth (parabuccal folds and “bitterness” wrinkles) and the eyes (crow's-feet), around which the cutaneous muscles are located, as well as between the eyebrows (glabellar furrows and frown lines) and on the forehead.

Thus, it is known that the cutaneous muscles of the face are under the control of afferent motor nerve impulses of the facial nerve and that, in addition, the interlobular septa of the subcutaneous tissue contain within them fibers which constitute a striated muscular tissue (panniculus carnosus). Moreover, it is also known that a subset of dermal fibroblasts, called myofibroblasts, display contractile characteristics that are common with the muscular tissue.

The means currently employed for acting upon laugh-lines are, on the one hand, botulinus toxin which is notably injected in the glabellar furrows (see J. D. Carruters et al., J. Dermatol. Surg. Oncol., 1992, 18, pp. 17-21) and, on the other hand, degradable implants based on collagen, hyaluronic acid or polylactic acid.

Moreover, as an alternative to these medical techniques requiring the services of a practitioner, various compounds that are able to provide a muscle relaxing effect when applied topically on the skin thus make it possible to exert an action on laugh-lines by another route. Among these compounds, we may notably mention antagonists of the receptors associated with calcium channels (FR-2 793 681), and in particular manganese and manganese salts (FR-2 809 005) and alverine (FR-2 798 590); and agonists of the receptors associated with the chlorine channels, including glycine (EP-0 704 210) and certain extracts of Iris pallida (FR-2 746 641).

However, there is still a need to find new agents for reducing these wrinkles and lines.

In particular, it would be advantageous to have at our disposal compounds that are able to act on various mechanisms involved in the appearance of the signs of ageing of the skin or of the mucosae, such as the appearance of wrinkles or of lines, thinning of the epidermis, or dehydration due to deterioration of the barrier function.

Unexpectedly, the applicant has now found that these and other aims are achieved by using ligands of the peripheral benzodiazepine receptors.

That is why the present invention relates to the cosmetic use of at least one antagonist of the peripheral benzodiazepine receptors in a composition containing a physiologically acceptable medium, as an agent for reducing or preventing the signs of ageing of the skin.

In fact, PBR antagonists according to the present invention can improve the appearance of the skin by promoting its hydration, improving its cellular renewal, promoting the synthesis of the constituents of the extracellular matrix and relaxing the tension of the cutaneous or subcutaneous contractile structures.

The pharmacological effects of the benzodiazepines (anxiolytic, anticonvulsant, muscle relaxant, and sedative) result from their attachment to the central benzodiazepine receptors (CBRs) present at the GABA-A (gamma-aminobutyric acid) receptor complex. GABA-A receptor denotes a macromolecular complex which, in addition to the GABA site, has sites for attachment of benzodiazepines, barbiturates, alcohol and certain steroids. It is a receptor that is associated with a channel having preferential permeability to chlorine ions (Cl⁻) and secondarily to bromine ions (Br⁻). Opening of this channel after attachment of the ligands described above leads to penetration of the Cl⁻ ions and hyperpolarization.

The CBRs are present exclusively in the central nervous system (CNS) and are localized on the neurons.

The benzodiazepines are also known to bind to peripheral receptors. These peripheral receptors are called peripheral benzodiazepine receptors (PBRs), whose structure differs significantly from that of the central receptors. They are said to be a major component of the protein complex localized on the membrane of the mitochondria, where they permit substances to pass through this membrane (MPTP or mitochondrial permeability transition pore); the mitochondrial benzodiazepine receptors (MBRs) are therefore representatives of the PBRs. PBRs have also been identified on erythrocytes and certain cancer cells.

DBI (diazepam binding inhibitor), a polypeptide of 11 kDa formed from 86 amino acids, is described as an endogenous ligand of the PBRS. DBI owes its name to the fact that it is able to inhibit the attachment of tritiated diazepam on the membranes of the brain and GABA_{A}-activated chlorine channel opening (Beurdeley-Thomas et al., “The peripheral benzodiazepine receptors: a review”, Journal of Neuro-Oncology, 46: 45-56, 2000; Zisterer et al., “Peripheral-type benzodiazepine receptors”, Gen. Pharmac. Vol. 29, No. 3, pp. 305-314, 1997).

The presence of PBRs on the sebaceous gland, and the capacity of the DBI for regulating the synthesis of cholesterol and of lipid in the sebaceous gland, have already been described in the document J. Invest. Dermatol., 1993, 101 (6), p. 800-803. The authors describe how DBI, an endogenous ligand of PBR, could, via its binding to acyl-CoA, stimulate cholesterol and lipid synthesis.

According to definitions that are well established in pharmacology, a molecule that, by binding to specific receptors, is able to trigger a biological action similar to the activity of the endogenous ligand, is by definition an agonist. Conversely, a molecule whose interaction with the same receptors is of a different nature, so that it is incapable of triggering the action-effect sequence (hence is without intrinsic activity) is by definition an antagonist (Schorderet et al., “Pharmacologie”, Frison-Roche et Slatkine Editions, 1992). As described in the document J. Invest. Dermatol. 1993, 101(6), p. 800-803, DBI is therefore a PBR agonist whose biological action leads to the synthesis of cholesterol and of lipid.

Antagonists of the PBRs and their use for preventing or treating diseases associated with dysfunction of the peripheral benzodiazepine receptors have already been described in the documents Le Fur, G. et al., Life Science, 1983, 33, 449-457 and in documents WO00/44384, WO02/07727, FR2811990, WO00/44751, WO03/082874, U.S. Pat. No. 6,767,533 describes the use of PBR ligands for protecting the skin against the effects of harmful stresses, such as UV exposure.

FR 2 811897 also describes the use of derivatives capable of binding to the peripheral benzodiazepine receptors, for the preparation of medicinal products intended for treating dysfunction of these receptors, in particular inflammatory diseases.

U.S. Pat. No. 5,976,559 relates to compositions containing a chlorine channel agonist, for combating wrinkles and lines.

In the present text, an antagonist of the peripheral benzodiazepine receptors (PBRs) is defined as any molecule of organic or inorganic origin having an affinity for the peripheral receptor binding sites for the benzodiazepines, and
(i) capable of reducing pregnenolone production induced by an agonist of PBR (MBR), and/or
(ii) whose affinity for the peripheral benzodiazepine receptors is defined by an entropy-dependent thermodynamic nature, and/or
(iii) promotes the regeneration of nerve cells and/or promotes cardiac recovery after ischemia and/or counters the growth of breast cancer cell lines.

The affinity for the peripheral receptor binding sites for the benzodiazepines can be measured in accordance with one of the binding studies described in documents WO00/44384, WO02/07727, FR2811990, WO00/44751, WO03/082874, WO03/068753 or WO03/030937.

The capacity for reducing pregnenolone production induced by a PBR (MBR) agonist can be measured according to the method described in documents

The entropy-dependent nature of the affinity for the peripheral benzodiazepine receptors can be measured according to the method described in document Le Fur, G. et al., Life Science, 1983, 33, 449-457.

In the present text, unless specified otherwise, skin is taken to mean the skin, the mucosae and/or the scalp.

“Cutaneous signs of ageing” means all changes in the outward appearance of the skin due to ageing whether it is chrono-biological and/or photo-induced, for example wrinkles and lines, withered skin, flabby skin, thin skin, lack of elasticity and/or tone of the skin, but also all internal changes of the skin which are not regularly reflected in an altered external appearance.

The invention notably relates to the use of an antagonist of the peripheral benzodiazepine receptors in a composition containing a physiologically acceptable medium, for reducing and/or preventing and/or delaying the signs of chronological ageing.

A physiologically acceptable medium is, according to the invention, a cosmetically or pharmaceutically acceptable medium, compatible with the skin, the mucosae, the nails and/or the hair.

The compositions according to the invention can be applied on the nails, the hair and more particularly on the skin and the mucosae. It is preferably a cosmetically acceptable medium, i.e. with a pleasant color, odor and feel, and which does not give rise to unacceptable discomfort.

The compositions are preferably cosmetic compositions or products. “Cosmetic product” means notably any substance or preparation intended to be brought into contact with the various surface regions of the human body (epidermis, hair on the body and head, nails, lips and external genital organs) or with the teeth and the oral mucosae for the purpose, exclusively or principally, of cleaning them, perfuming them, modifying their appearance and/or correcting body odors and/or protecting them or keeping them in a good condition (cosmetic directive 76/768/EEC as amended).

Within the scope of the invention, it was found that antagonists of the peripheral benzodiazepine receptors reduce the differentiation of the keratinocytes. This effect is assessed notably by measuring the activity of transglutaminase K1 (TGK1). In fact, the epidermis is constantly being renewed. The numerous cells produced in the basal layer may either remain quiescent, or be eliminated by apoptosis, or take part in a process of differentiation in order to reconstitute the suprabasal layers of the epidermis. The final stage of epidermal differentiation is cornification of the last layers of living cells. This cornification takes place owing to the presence of specific enzymes such as transglutaminase K1 which participates in the formation of molecular bridges between the various precursors synthesized in the living layers (involucrin, loricrin, keratolinin). This enzymatic activity therefore reflects terminal differentiation of the epidermis.

Unexpectedly, it has now been found that the production of transglutaminase K1 (TGK1) is reduced in the presence of an antagonist according to the invention. Such activity, similar to that of retinol, counters differentiation of the epidermis; these compounds improve the radiance of the complexion, by thickening the epidermis and limiting the thickness of the horny layer.

Such effects are particularly advantageous for the skin of the face, and notably for limiting the loss of radiance due to age.

According to another aspect of the invention, the antagonists of the peripheral benzodiazepine receptors can be used for promoting the synthesis of the constituents of the extracellular matrix, especially the epidermal macromolecules. The extracellular matrix is an assemblage of structural molecules deposited and organized by the cells. These molecules are, in their turn, capable of exerting an influence on cell behavior. In the epidermis, despite the presence of many intercellular junctions and a limited intercellular space, the presence of matrix elements (fibronectin, proteoglycans and glycosaminoglycans) suggests their potential roles in various processes such as nutrition and hydration of the epidermis, influence on cell behavior (migration, epidermal differentiation). In particular, the PBR antagonists according to the invention can be used for increasing the quantity of total epidermal glycosaminoglycans; they act notably by increasing their synthesis by the keratinocytes.

The antagonists of the peripheral benzodiazepine receptors can therefore be used according to the invention for improving the hydration of the epidermis, by improving the barrier function and by promoting the maintenance of water in the surface layers of the skin. This activity will be advantageous in all cases where the skin is dry and/or dehydrated. It is known that the skin tends to dry out with age, and one of the applications will therefore notably be the treatment of dehydration of the skin, whether associated with age or other causes, notably if it is part of one's constitution.

According to yet another aspect of the invention, the antagonists of the peripheral benzodiazepine receptors can be used as agents for relaxing the cutaneous and/or subcutaneous tissue. They are particularly suitable for combating wrinkles and lines, especially for preventing and/or reducing laugh-lines.

Thus, the antagonists of the peripheral benzodiazepine receptors are active, in accordance with the present invention, both at the level of the dermis and of the epidermis, and make it possible to combat a number of signs of chronological ageing.

According to one embodiment of the invention, the antagonists of the peripheral benzodiazepine receptors can be used as agents for combating age-related thinning of the skin, flabby and withered skin, and/or for combating lack of tone or elasticity of the skin.

Preferably, the PBR antagonists according to the invention are:

(A) PK11195 (or 1-(2-chlorophenyl)-N-(1-methylpropyl)-3-isoquinoline carboxamide) as described in the document Le Fur, G. et al., Life Science, 1983, 33, 449-457;
(B) the compounds of the following general formula (I) as described in document WO03/030937:

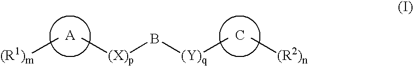

in which:
A and C each represent, independently of one another, a ring or a carbon-containing heterocycle with 5 to 10 ring members;
B represents

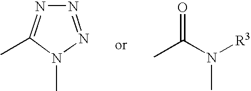

in which R³ represents a hydrogen atom; a C₁-C₈ alkyl optionally substituted with a phenyl group; a C₂-C₈ acyl group optionally substituted with a phenyl group; a C₁-C₈ alkoxycarbonyl; or
a C₁-C₄ alkylene,
X and Y each represent, independently of one another, —CH₂—, —O—, or —CHR⁴— in which R⁴ represents a C₁-C₄ alkyl, or a C₂-C₅ alkylene,
p and q each represent, independently of one another, an integer equal to 0 or varying from 2 to 4, it being understood that p and q are not simultaneously equal to 0,
R¹ and R² each represent, independently of one another:
   1) a halogen atom,

### 2) OR⁵,

### 3) SR⁵,

### 4) NR⁶R⁷,

5) a nitro,
6) a cyano,

### 7) COR⁸,

8) a ring or a carbon-containing heterocycle with 3 to 10 ring members, optionally substituted with 1 to 5 groups selected from a C₁-C₈ alkyl, said alkyl being optionally substituted with 1 to 5 groups selected from a halogen atom, OR⁵, SR⁵, NR⁶R⁷, a nitro group, a cyano group, COR⁸ and a phenyl; a halogen atom; OR⁵; SR⁵; NR⁶R⁷; a nitro; a cyano; COR⁸ and a ring or a carbon-containing heterocycle with 5 to 10 ring members, said ring or heterocycle being optionally substituted with 1 to 5 groups selected from a halogen atom, OR⁵, SR⁵, NR⁶R⁷, a nitro group, a cyano group, COR⁸ and a phenyl and a C₁-C₈ alkyl, or
9) a C₁-C₈ alkyl, optionally substituted with 1 to 5 groups selected from a halogen atom, OR⁵, SR⁵, NR⁶R⁷, a nitro, a cyano, COR⁸ and a ring or a carbon-containing heterocycle with 5 to 10 ring members, said ring or heterocycle being optionally substituted with 1 to 5 groups selected from a halogen atom, OR⁵, SR⁵, NR⁶R⁷, a nitro, a cyano, COR⁸ and a phenyl and a C₁-C₈ alkyl, said alkyl being optionally substituted with 1 to 5 groups selected from a halogen atom, OR⁵, SR⁵, NR⁶R⁷, a nitro, a cyano, COR⁸ and a phenyl;
in which:
R⁵ represents:
   1) a hydrogen atom;
   2) a C₁-C₈ alkyl, optionally substituted with 1 to 5 groups selected from a halogen atom, OR⁹, SR⁹, NR¹⁰R¹¹, a nitro, a cyano, COR¹² and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², a phenyl and a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl;
   3) a ring or a carbon-containing heterocycle with 3 to 10 ring members, optionally substituted with 1 to 5 groups selected from a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl; a halogen atom; OR⁹; SR⁹; NR¹⁰R¹¹; a nitro; a cyano; COR¹²; a phenyl and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², a phenyl and a C₁-C₈ alkyl;
   4) COR¹³, in which R¹³ represents a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl; or a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², a phenyl and a C₁-C₈ alkyl, and a C₁-C₈ alkyl;
R⁶ and R⁷ each represent, independently of one another:
   1) a hydrogen atom;
   2) a C₂-C₈ acyl, optionally substituted with 1 to 5 groups selected from a halogen atom, OR⁹, SR⁹, NR¹⁰R¹¹, a nitro, a cyano, COR¹² and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², and a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl;
   3) COOR¹²;
   4) CONR¹⁰R¹¹;
   5) a ring or a carbon-containing heterocycle with 3 to 10 ring members, optionally substituted with 1 to 5 groups selected from a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl; a halogen atom; OR⁹; SR⁹; NR¹⁰R¹¹; a nitro; a cyano; COR¹²; a phenyl and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², and a C₁-C₈ alkyl, said alkyl being optionally substituted with 1 to 5 groups selected from a halogen atom, OR⁹, SR⁹, NR¹⁰R¹¹, a nitro, a cyano, COR¹²; or
   6) a C₁-C₈ alkyl, optionally substituted with 1 to 5 groups selected from a halogen atom, OR⁹, SR⁹, NR¹⁰R¹¹, a nitro, a cyano, COR¹² and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², and a C₁-C₈ alkyl, said alkyl being optionally substituted with 1 to 5 groups selected from a halogen atom, OR⁹, SR⁹, NR¹⁰R¹¹, a nitro, a cyano, COR¹²;
R⁸ represents
   1) a hydrogen atom;
   2) OR⁹;
   3) NR¹⁰R¹¹;
   4) a ring or a carbon-containing heterocycle with 3 to 10 ring members, optionally substituted with 1 to 5 groups selected from a halogen atom; OR⁹; SR⁹; NR¹⁰R¹¹; a nitro; a cyano; COR¹²; a phenyl; a C₁-C₈ alkyl, said alkyl being optionally substituted with 1 to 5 groups selected from a halogen atom, OR⁹, SR⁹, NR¹⁰R¹¹, a nitro, a cyano, COR¹² and a phenyl; and a ring or a carbon-containing heterocycle with 3 to 10 ring members, optionally substituted with 1 to 5 groups selected from a halogen atom, OR⁹, SR⁹, NR¹⁰R¹¹, a nitro, a cyano, COR¹², a C₁-C₈ alkyl and a phenyl; or
   5) a C₁-C₈ alkyl, optionally substituted with 1 to 5 groups selected from a halogen atom, OR⁹, SR⁹, NR¹⁰R¹¹, a nitro, a cyano, COR¹² and a ring or a carbon-containing heterocycle with 3 to 10 ring members, said ring or heterocycle being optionally substituted with 1 to 5 groups selected from a halogen atom, OR⁹, SR⁹, NR¹⁰R¹¹, a nitro, a cyano, COR¹², and a C₁-C₈ alkyl, said alkyl being optionally substituted with 1 to 5 groups selected from a halogen atom, OR⁹, SR⁹, NR¹⁰R¹¹, a nitro, a cyano, COR¹², and a phenyl;
   in which:
      R⁹ represents a hydrogen atom, a C₁-C₈ alkyl, a C₂-C₈ acyl, said alkyl or acyl being optionally substituted with a C₁-C₈ alkoxy, a C₁-C₈ alkylthio, or a ring or a carbon-containing heterocycle with 3 to 10 ring members,
      R¹⁰ and R¹¹ each represent, independently of one another, a hydrogen atom, a C₁-C₈ alkyl, or a phenyl, and
      R¹² represents a hydrogen atom, a phenyl, a C₁-C₈ alkyl optionally substituted with a phenyl, and a C₁-C₈ alkoxy optionally substituted with a phenyl, and
      m and n each represent, independently of one another, an integer equal to 0 or varying from 1 to 5.
         (C) the compounds of the following general formula (II) as described in document WO03/030937:

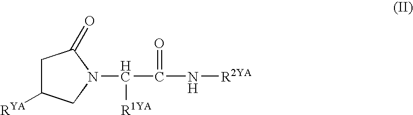

in which:
R^{YA} represents a hydrogen atom or a hydroxyl group,
R^{1YA} represents a hydrogen atom or a methyl,
R^{2YA} represents a pyridyl or a phenyl substituted with 1 to 3 groups, which may be identical or different, selected from:
   a halogen atom,
   a trifluoromethyl,
   a nitro,
   an acetyl,
   a linear or branched C₁-C₄ alkyl,
   a linear or branched C₁-C₄ alkoxy,
   a linear or branched C₁-C₇ alkylmercapto,
   a substituted alkylmercapto of general formula

—S—(CH₂)_{nYA}—CH(R^{3YA})(R^{4YA}),
in which:
   n^{YA} represents an integer varying from 1 to 2,
   R^{3YA} represents a hydrogen atom or a methyl, and
   R^{4YA} represents a hydroxyl group; an amine of general formula —NR^{8YA}R^{9YA}, in which:
      R^{8YA} represents a hydrogen atom or a methyl,
      R^{9YA} represents a methyl, a benzyl or a substituted benzyl, or
      R^{8YA} and R^{9YA}, taken together with the nitrogen atom carrying them, form a substituted pyrrolidine ring,
a sulfonyl of general formula —SO₂R^{5YA}, in which R^{5YA} represents an amine or a C₁-C₃ alkyl, and
an aminoethoxycarbonyl of general formula —COO(CH₂)₂—NR^{6YA}R^{7YA}, in which R^{6YA} and R^{7YA} each represent, independently of one another, a hydrogen atom, a methyl or an ethyl.
   (D) the compounds of the following general formula (III) as described in document WO03/030937:

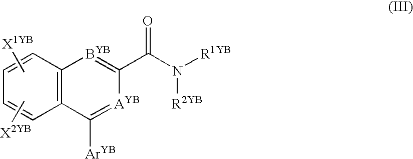

in which:
R^{1YB} and R^{2YB} each represent, independently of one another, a linear or branched C₁-C₆ alkyl; a C₃-C₇ cycloalkyl; a C₁-C₃ alkyl substituted with a phenylalkyl or a cycloalkyl; a C₃-C₆ alkenyl or an alkynyl, it being understood that the double bond of said alkenyl or the triple bond of said alkynyl is not in position 1 or 2 relative to the nitrogen atom;
A^{YB} and B^{YB} each represent, independently of one another, a nitrogen atom or a CH group;
X^{1YB} and X^{2YB} each represent, independently of one another, a halogen atom, a linear or branched C₁-C₃ alkyl, a linear or branched C₁-C₃ alkoxy, a nitro or a trifluoromethyl; and
Ar^{YB} represents a phenyl, a pyridyl, a thienyl or a phenyl substituted with one or two groups, each independently of one another, selected from a halogen atom, a linear or branched C₁-C₄ alkyl, a linear or branched C₁-C₄ alkoxy, a linear or branched C₁-C₄ alkylthio, a trifluoromethyl and a nitro.
   (E) the compounds of the following general formula (IV) as described in document WO03/030937:

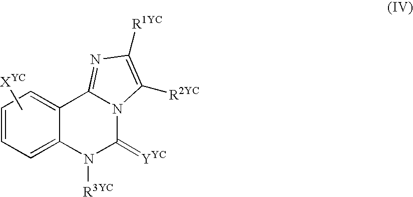

in which:
R^{1YC} represents an unsubstituted phenyl, a phenyl substituted with one or two groups, each independently of one another, selected from a halogen atom, a linear or branched C₁-C₆ alkyl, a linear or branched C₁-C₆ alkoxy; or a thienyl;
R^{2YC} represents a hydrogen atom, a linear or branched alkyl having a number of carbon atoms varying from 1 to 6 and optionally a nitrogen atom and/or a substituent selected from an amino, an alkylamino or a dialkylamino;
R^{3YC} represents a group with the general formula (R^{4YC})(R^{5YC})N—CO-Q^{YC}, in which:
   Q^{YC} represents a linear or branched C₁-C₆ alkyl,
   R^{4YC} and R^{5YC} each represent, independently of one another, a linear or branched C₁-C₆ alkyl, a phenyl, or a phenyl substituted with one or two groups, each independently of one another, selected from a halogen atom, a linear or branched C₁-C₆ alkyl, a linear or branched C₁-C₆ alkoxy;
X^{YC} represents a hydrogen atom or a halogen atom;
Y^{YC} represents an oxygen atom or a sulfur atom.
   (F) the compounds of the following general formula (V) as described in document WO03/030937:

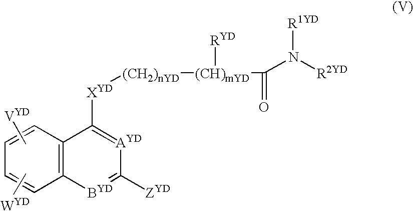

in which:
A^{YD} and B^{YD} each represent, independently of one another, a nitrogen atom or a CH group;
V^{YD} and W^{YD} each represent, independently of one another, a hydrogen atom, a halogen atom, a C₁-C₃ alkyl, a C₁-C₃ alkoxy, a nitro or a trifluoromethyl;
Z^{YD} represents a phenyl, a thienyl or a pyridyl, said phenyl being unsubstituted or substituted with one or two groups, each independently of one another, selected from a C₁-C₄ alkyl, a C₁-C₄ alkoxy, a trifluoromethyl or a nitro;
R^{YD} represents a hydrogen atom or a C₁-C₃ alkyl;
R^{1YD} and R^{2YD} each represent, independently of one another, a C₁-C₆ alkyl; a C₃-C₆ cycloalkyl, a phenyl, a C₁-C₃ phenylalkyl, a C₃-C₆ cycloalkyl-C₁-C₃ alkyl group or a C₃-C₆ alkenyl, it being understood that the double bond of the alkenyl is not in position 1 or 2 relative to the nitrogen atom; or NR^{1YD}R^{2YD} represents a pyrrolidine, a piperidine, a morpholine or a thiomorpholinone;
X^{YD} represents CHR^{3YD}, NR^{YD}, SO, SO₂, O or S;
R^{3YD} represents a hydrogen atom or a C₁-C₃ alkyl;
R^{4YD} represents a C₁-C₃ alkyl;
m^{YD} represents an integer equal to 0 or 1;
n^{YD} represents an integer equal to 0 or varying from 1 to 2;
it being understood that:
(1) if X^{YD} represents SO, SO₂ or NR^{4YD}, then m^{YD}+n^{YD} is at least equal to 1,
(2) if A^{YD} and B^{YD} each represent a nitrogen atom, Z^{YD} is in the para position relative to B^{YD}, then X^{YD} does not represent CHR^{3YD},
(3) if Z^{YD} represents a CH group, B^{YD} represents a nitrogen atom, Z^{YD} is in the ortho position relative to B^{YD}, X^{YD} represents an oxygen atom, and R^{YD} represents a hydrogen atom, then m^{YD}+n^{YD} is not equal to 1, and
(4) 2-phenylquinolin-4-yl-N,N′-dimethylcarbamate is excluded.

The compounds of formulas (I) to (V) can be prepared according to the methods of synthesis described in document WO03/030937.

(G) the compounds of the following general formula (VI) or a pharmacologically acceptable salt thereof as described in document WO03/068753:

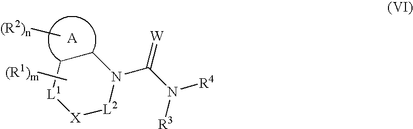

in which:
ring A represents a C₅-C₈ carbon-containing monocycle or a heterocyclic monocycle with 5 to 8 ring members comprising 1 to 2 nitrogen atoms, 1 to 2 oxygen atoms and/or a sulfur atom,
X represents (1) —CH₂—, (2) —O—, (3) —S—, (4) —S(O)— or (5) —SO₂—,
L¹ and L² each represent, independently of one another, a saturated C₁-C₄ alkylene group, a C₂-C₄ alkylene group or alkenylene group, in which the total number of carbons in L¹ and L² is 3 or 4,
R¹ and R² each represent, independently of one another:
   (1) a C₁-C₈ alkyl group, a C₂-C₈ alkenyl group, a C₂-C₈ alkynyl group, optionally substituted with 1 to 5 groups selected from ring B, OR⁵, NR⁶R⁷, COR⁸, OCOR⁸, OCONR⁶R⁷, COOR⁸, SR⁹, SOR⁸, SO₂R⁸, SO₂NR⁶R⁷, a halogen atom, a carboxyl group, a cyano group and a nitro group, (2) ring B, (3) OR⁵, (4) NR⁶R⁷, (5) COR⁸, (6) OCOR⁸, (7) OCONR⁶R⁷, (8) COOR⁸, (10) SR⁹, (11) SOR⁸, (12) SO₂R⁸, (13) SO₂NR⁶R⁷, (14) a halogen atom, (15) a carboxyl group, (16) a cyano group, (17) a nitro group, (18) an oxo group, or (19)

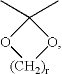

in which:
ring B represents (i) a monocyclic or bicyclic C₃-C₁₀ carbon-containing ring, or (ii) a monocyclic or bicyclic heterocycle with 5 to 10 ring members comprising 1 to 2 nitrogen atoms, 1 to 2 oxygen atoms and/or a sulfur atom, said ring B is optionally substituted with 1 to 5 groups selected from (i) a C₁-C₈ alkyl group, a C₂-C₈ alkenyl group, a C₂-C₈ alkynyl group, optionally substituted with 1 to 5 groups selected from ring B, OR⁵, NR⁶R⁷, COR⁸, OCOR⁸, OCONR⁶R⁷, COOR⁸, SR, SOR⁸, SO₂R⁸, SO₂NR⁶R⁷, a halogen atom, a carboxyl group, a cyano group and a nitro group, (ii) OR⁵, (iii) NR⁶R⁷, (iv) COR⁸, (v) OCOR⁸, (vi) OCONR⁶R⁷, (vii) COOR⁸, (viii) CONR⁶R⁷, (ix) SR⁸, (x) SOR⁸, (xi) SO₂R⁸, (xii) SO₂NR⁶R⁷, (xiii) a halogen atom, (xiv) a carboxyl group, (xv) a cyano group, (xvi) a nitro group, (xvii) an oxo group,
R⁵ represents (i) a hydrogen atom, (ii) a C₁-C₈ alkyl group, a C₂-C₈ alkenyl group, a C₂-C₈ alkynyl group, optionally substituted with 1 to 5 groups selected from ring B, OR¹⁵, NR¹⁶R¹⁷, COR¹⁸, OCOR¹⁸, OCONR¹⁶R¹⁷, COOR¹⁸, SR¹⁹, SOR⁸, SO₂R⁸, SO₂NR⁶R⁷, ring C, a halogen atom, a carboxyl group, a cyano group and a nitro group, (iii) —Si (R¹⁰)₃ or (iv) ring C,
R⁶ and R⁷ each represent, independently of one another, (i) a hydrogen atom, or (ii) a group -D¹-D², in which:
   D¹ represents (a) a single bond, (b) —C(O)—, (c) —C(O)O or (d) —SO₂—;
   D² represents (a) a C₁-C₈ alkyl group, a C₂-C₈ alkenyl group, a C₂-C₈ alkynyl group, optionally substituted with ring C, or (b) ring C, said ring C represents (a) a mono- or bicyclic C₃-C₁₀ carbon-containing ring or (b) a mono- or bicyclic heterocycle with 5 to 10 ring members comprising 1 to 2 nitrogen atoms, 1 to 2 oxygen atoms and/or a sulfur atom, said ring C is optionally substituted with 1 to 5 groups selected from a C₁-C₈ alkyl group, OR¹⁵, NR¹⁶R¹⁷, COR¹⁸, OCOR¹⁸, OCONR¹⁶R¹⁷, COOR¹⁸, SR¹⁹, SOR⁸, SO₂R⁸, SO₂NR⁶R⁷, ring C, a halogen atom, a carboxyl group, a cyano group and a nitro group, an oxo group,
R⁸ represents (i) a C₁-C₈ alkyl group, a C₂-C₈ alkenyl group, a C₂-C₈ alkynyl group, optionally substituted with ring C, or (ii) ring C,
R⁹ represents (i) a hydrogen atom, (ii), a C₁-C₈ alkyl group, a C₂-C₈ alkenyl group, a C₂-C₈ alkynyl group, optionally substituted with at least one group selected from OR¹⁵, NR¹⁶R¹⁷, COR¹⁸, OCOR¹⁸, OCONR¹⁶R¹⁷, COOR¹⁸, SR¹⁹, SOR⁸, SO₂R⁸, SO₂NR⁶R⁷, a halogen atom, or (iii) ring B,
the plurality of R¹⁰ represents, independently of one another, a C₁-C₈ alkyl group or a phenyl group,
R¹⁵ and R¹⁹ each represent, independently of one another, a C₁-C₈ alkyl group, a C₂-C₈ alkenyl group, a C₂-C₈ alkynyl group, a C₂-C₈ acyl group,
R¹⁶ and R¹⁷ each represent, independently of one another, (ii) a C₁-C₈ alkyl group, a C₂-C₈ alkenyl group, a C₂-C₈ alkynyl group, (ii) a phenyl group, optionally substituted with a C₁-C₈ alkyl group, a C₂-C₈ alkenyl group, a C₂-C₈ alkynyl group, a halogen atom, a C₂-C₈ alkoxy group or a C₂-C₈ alkenyloxy group,
   R¹⁸ represents a C₁-C₈ alkyl group, a C₂-C₈ alkenyl group, a C₂-C₈ alkynyl group and,
   r represents an integer varying from 2 to 4,
m and n each represent, independently of one another, an integer equal to 0 or varying from 2 to 4,
R³ represents (i) a hydrogen atom, (ii) ring B, or (iii) a C₁-C₈ alkyl group, a C₂-C₈ alkenyl group, a C₂-C₈ alkynyl group, optionally substituted with 1 to 5 groups selected from ring B, OR⁵, NR⁶R⁷, COR⁸, OCOR⁸, OCONR⁶R⁷, COOR⁸, CONR⁶R⁷, SR, SOR⁸, SO₂R⁸, SO₂NR⁶R⁷, a halogen atom, a carboxyl group, a cyano group and a nitro group,
R⁴ represents a hydrogen atom, a C₁-C₈ alkyl group, a C₂-C₈ alkenyl group, a C₂-C₈ alkynyl group,
R³ and R⁴ can optionally form, together with the nitrogen atom carrying them, a mono- or bicyclic heterocycle with 5 to 10 ring members containing a nitrogen atom, and optionally from 1 to 3 other nitrogen atoms, an oxygen atom and/or a sulfur atom, said heterocycle being optionally substituted with 1 to 5 groups selected from a C₁-C₈ alkyl, a group OR¹⁵, NR¹⁶R¹⁷, COR¹⁸, OCOR¹⁸, OCNR¹⁶R¹⁷, COOR¹⁸, SR¹⁹, SOR⁸, SO₂R⁸, SO₂NR⁶R⁷, a halogen atom, a carboxyl group, a cyano group and a nitro group, an oxo group.

The compounds of formula (VI) can be prepared according to the methods of synthesis described in document WO03/068753.

(H) the derivatives of pyridazino[4,5-b]indole-1-acetamide of general formula (VII) as described in document WO00/44384:

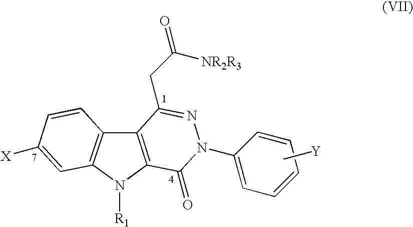

in which:
X represents a halogen atom,
Y represents one or more atoms or groups selected from hydrogen, the halogens and the hydroxy, methyl, methoxy and nitro groups,
R₁ represents a (C₁-C₄) alkyl group,
R₂ and R₃ each represent, independently of one another, a hydrogen atom or a (C₁-C₄) alkyl group, or alternatively R₂ and R₃ form, with the nitrogen atom carrying them, a pyrrolidinyl, piperidinyl or morpholinyl group.

The compounds of formula (VII) can be prepared according to the methods of synthesis described in document WO00/44384.

Advantageously, the compounds of formula (VII) are selected from compounds 1 to 34 in the following Table 1.

TABLE 1

| No. | X | Y | R₁ | NR₂R₃ |
|---|---|---|---|---|
| 1 | Cl | H | Me | NMe₂ |
| 2 | Cl | H | Me | NEt₂ |
| 3 | Cl | H | Me | Pyrrolid |
| 4 | Cl | H | Me | Morph |
| 5 | Cl | 3-Me | Me | NMe₂ |
| 6 | Cl | 3-Me | Me | NEt₂ |
| 7 | Cl | 3-Me | Me | Pyrrolid |
| 8 | Cl | 3-Cl | Me | NMe₂ |
| 9 | Cl | 3-Cl | Me | NEt₂ |
| 10 | Cl | 3-Cl | Me | Pyrrolid |
| 11 | Cl | 3-Cl | Me | Piperidi |
| 12 | Cl | 2-Cl | Me | NMe₂ |
| 13 | Cl | 2-Cl | Me | NEt₂ |
| 14 | Cl | 2-Cl | Me | Pyrrolid |
| 15 | Cl | 4-Cl | Me | NMe₂ |
| 16 | Cl | 4-Cl | Me | NEt₂ |
| 17 | Cl | 4-Cl | Me | Pyrrolid |
| 18 | Cl | 3-OMe | Me | NMe₂ |
| 19 | Cl | 3-OMe | Me | NEt₂ |
| 20 | Cl | 3-OMe | Me | Pyrrolid |
| 21 | Cl | 3-NO₂ | Me | NMe₂ |
| 22 | Cl | 3-NO₂ | Me | NEt₂ |
| 23 | Cl | 3-NO₂ | Me | Pyrrolid |
| 24 | Cl | 3-F | Me | NMe₂ |
| 25 | Cl | 3-F | Me | NEt₂ |
| 26 | Cl | 3-F | Me | Pyrrolid |
| 27 | Cl | 3,5-(Cl)₂ | Me | NEt₂ |
| 28 | Cl | 4-Cl | Me | NMeEt |
| 29 | Cl | H | Me | NHMe |
| 30 | Cl | H | Me | NH₂ |
| 31 | Cl | 4-OMe | Me | NMe₂ |
| 32 | Cl | 4-OMe | Me | NEt₂ |
| 33 | Cl | 4-OH | Me | NMe₂ |
| 34 | Cl | 4-OH | Me | NEt₂ |

“Me” and “Et” denote, respectively, a methyl group and an ethyl group.

“Pyrrolid”, “Piperid” and “Morph” denote, respectively, a pyrrolidinyl, piperidinyl and morpholinyl group.

(I) the derivatives of 1-(4-oxo-3,5-dihydro-4H-pyridazino[4,5-b]indole-1-carbonyl)piperazine of formula (VIII) as described in document FR2811990:

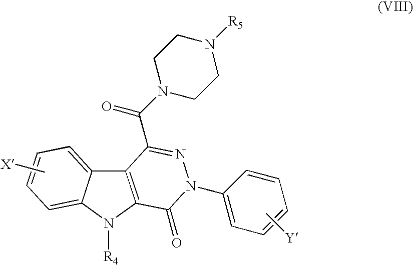

in which:
X¹ represents a hydrogen atom or a halogen atom,
Y′ represents one or more atoms or groups selected from hydrogen, the halogens and the methyl, hydroxy and methoxy groups,
R₄ represents a hydrogen atom or a (C₁-C₄) alkyl group,
R₅ represents a hydrogen atom, a linear, branched or cyclic (C₁-C₆) alkyl group, a (C₃-C₇) cycloalkyl (C₁-C₆) alkyl group, a phenyl group, a pyridinyl group or a phenylmethyl group.
   The compounds of general formula (VIII) can exist as bases or as salts of addition to acids.
   The compounds of formula (VIII) can be prepared according to the methods of synthesis described in document FR2811990.

Advantageously, the compounds of formula (VIII) are selected from compounds 35 to 52 in the following Table

TABLE 2

| No. | X′ | Y′ | R₄ | R₅ | Salt |
|---|---|---|---|---|---|
| 35 | 7-Cl | H | Me | H | HCl 1:1 |
| 36 | 7-Cl | H | Me | CH₃ | HCl 1:1 |
| 37 | 7-Cl | H | Me | CH₂CH₃ | HCl 1:1 |
| 38 | 7-Cl | H | Me | (CH₂)₂CH₃ | HCl 1:1 |
| 39 | 7-Cl | H | Me | CH(CH₃)₂ | HCl 1:1 |
| 40 | 7-Cl | H | Me | cC₆H₁₁ | HCl 1:1 |
| 41 | 7-Cl | H | Me | C₆H₅ | — |
| 42 | 7-Cl | H | Me | CH₂C₆H₅ | HCl 1:1 |
| 43 | 7-Cl | H | Me | 2-C₅H₄N | HCl 1:1 |
| 44 | 7-Cl | 4-C1 | Me | CH₃ | HCl 1:1 |
| 45 | 7-Cl | 3-C1 | Me | CH₃ | HCl 1:1 |
| 46 | 7-Cl | 2-C1 | Me | CH₃ | HCl 1:1 |
| 47 | 8-F | H | Me | CH₃ | HCl 1:1 |
| 48 | 8-F | H | Me | CH(CH₃)₂ | HCl 1:1 |
| 49 | 8-F | H | Me | cC₆H₁₁ | HCl 1:1 |
| 50 | H | H | Me | CH₃ | HCl 1:1 |
| 51 | H | H | Me | cC₆H₁₁ | HCl 1:1 |
| 52 | H | H | Me | CH(CH₃)₂ | HCl 1:1 |

In column “R₅”, “C₆H₅” denotes a phenyl group, “cC₆H₁₁” denotes a cyclohexyl group and “C₅H₄N” denotes a pyridin-2-yl group. In the column “Salt”, “-” denotes a compound in the base state and “HCl” denotes a hydrochloride; the acid:base molar ratio is shown alongside.

(J) the derivatives of pyridazino(4,5)indole-1-acetamide of formula (IX) as described in document WO02/07727:

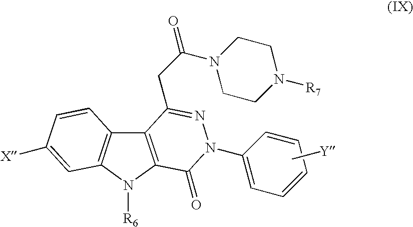

in which:
X″ represents a halogen atom,
Y″ represents one or more atoms or groups selected from hydrogen, the halogens and the hydroxy, methyl and methoxy groups,
R₆ represents a hydrogen atom or a (C₁-C₄)alkyl group,
R₇ represents a hydrogen atom, a linear or branched (C₁-C₄)alkyl group, a hydroxy(C₁-C₄)alkyl group, a (C₃-C₇)cycloalkyl group, a (C₁-C₆)alkyl group, a phenyl group, a pyridinyl group or a phenyl (C₁-C₄) alkyl group.

The compounds of general formula (IX) can exist as bases or as salts of addition to acids.

The compounds of formula (IX) can be prepared according to the methods of synthesis described in document WO02/07727.

Advantageously, the compounds of formula (IX) are selected from compounds 53 to 66 in the following Table 3.

TABLE 3

| No. | X″ | Y″ | R₆ | R₇ | Salt |
|---|---|---|---|---|---|
| 53 | Cl | H | Me | H | HCl 1:1 |
| 55 | Cl | H | Me | CH₃ | HCl 1:1 |
| 56 | Cl | H | Me | CH₂CH₃ | HCl 1:1 |
| 57 | Cl | H | Me | (CH₂)₂CH₃ | HCl 1:1 |
| 58 | Cl | H | Me | CH(CH₃)₂ | HCl 1:1 |
| 59 | Cl | H | Me | cC₆H₁₁ | HCl 1:1 |
| 60 | Cl | H | Me | C₆H₅ | — |
| 61 | Cl | H | Me | CH₂C₆H₅ | HCl 1:1 |
| 62 | Cl | H | Me | 2-C₅H₄N | HCl 1:1 |
| 63 | Cl | H | Me | CH₂cC₃H₅ | HCl 1:1 |
| 64 | Cl | H | Me | 3-C₅H₄N | HCl 1:1 |
| 65 | Cl | 3-F | Me | CH₃ | HCl 1:1 |
| 66 | Cl | H | Me | CH₂CH₂OH | HCl 1:1 |

In column “R₇”, “cC₃H₅” denotes a cyclopropyl group, cC₆H₁₁ denotes a cyclohexyl group, C₆H₅ denotes a phenyl group and 2-C₅H₄N denotes a pyridin-2-yl group. In column “Salt”, “-” denotes a compound in the base state and “HCl” denotes a hydrochloride; the acid:base molar ratio is shown alongside.

(K) the derivatives of 3-hereoaryl-3,5-dihydro-4-oxo-4H-pyridazino[4,5-b]indolel-acetamide of formula (X) as described in document WO03/082874.

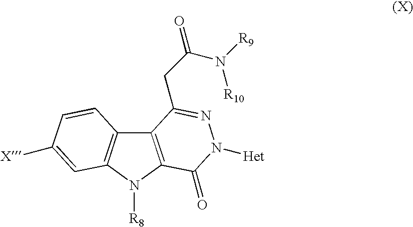

in which:
X′″ represents a halogen atom,
R₈ represents a hydrogen atom or a (C₁-C₄)alkyl group,
R₉ and R₁₀ each represent, independently of one another, a hydrogen atom, a linear (C₁-C₄)alkyl group or alternatively R⁹ and R¹⁰ form, with the nitrogen atom carrying them, a pyrrolidinyl, piperidinyl or morpholinyl or 4-(C₁-C₄)alkylpiperazinyl group, and
Het represents a heteroaromatic group of the pyridinyl, quinolinyl, isoquinolinyl, pyrimidinyl, pyrazinyl, pyridazinyl group, and the heteroaromatic group can carry one or more halogen atoms and/or one or more (C₁-C₄)alkyl or (C₁-C₄)alkoxy groups.

The compounds of general formula (X) can exist as bases or as salts of addition to acids, as well as the hydrate or solvate.

The compounds of formula (X) can be prepared according to the methods of synthesis described in document WO03/082874.

Advantageously, the compounds of formula (X) are selected from compounds 67 to 78 in the following Table 4.

TABLE 4

| No. | X′′′ | R₈ | NR₉R₁₀ | Het | Salt |
|---|---|---|---|---|---|
| 67 | F | CH₃ | N(CH₃)₂ | pyridin-2-yl | — |
| 68 | F | CH₃ | N(CH₃)₂ | pyridin-3-yl | HCl 1:1 |
| 69 | F | CH₃ | N(CH₃)₂ | pyridin-4-yl | HCl 1:1 |
| 70 | Cl | CH₃ | N(CH₃)₂ | pyridin-2-yl | — |
| 71 | Cl | CH₃ | N(CH₃)₂ | pyridin-3-yl | — |
| 72 | Cl | CH₃ | N(CH₃)₂ | pyridin-3-yl | HCl 1:1 |
| 73 | Cl | CH₃ | N(CH₃)₂ | pyridin-4-yl | HCl 1:1 |
| 74 | Cl | CH₃ | N(CH₃)₂ | 6-methylpyridin-3-yl | — |
| 75 | Cl | CH₃ | N(CH₂CH₃)₂ | pyridin-3-yl | — |
| 76 | Cl | CH₃ | 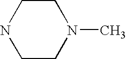 | pyridin-3-yl | HCl 1:1 |
| 77 | Cl | CH₃ | 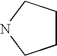 | pyridin-3-yl | — |
| 78 | Cl | CH₃ | 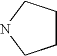 | pyridin-4-yl | HCl 1:1 |

The compounds of formulas (I), (II), (III), (IV) and (V) that are preferred according to the invention are selected from the compounds of the following formulas (I-a-1) to (I-a-32) and (I-b-1) to (I-b-32)

| | |
|---|---|
| | (I-a-1) |
| 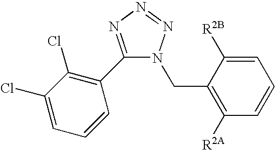 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-2) |
| 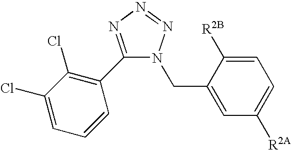 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-3) |
| 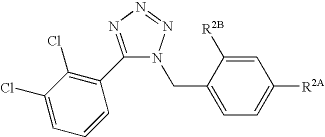 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-4) |
| 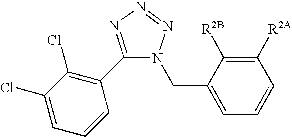 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-5) |
| 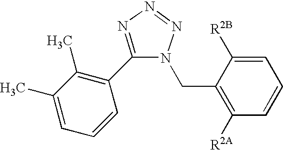 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₂Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-6) |
| 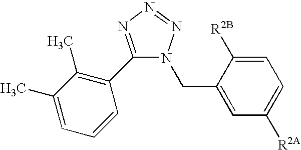 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-7) |
| 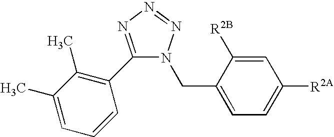 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-8) |
| 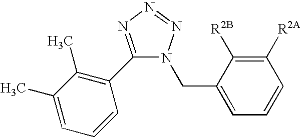 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-9) |
| 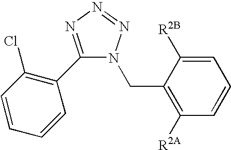 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-10) |
| 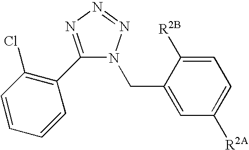 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-11) |
| 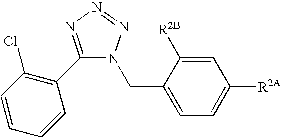 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-12) |
| 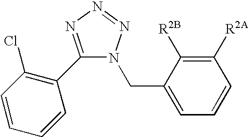 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-13) |
| 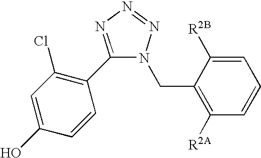 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-14) |
| 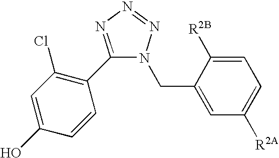 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-15) |
| 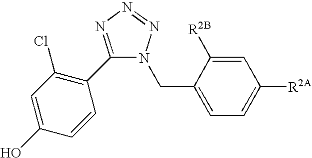 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-16) |
| 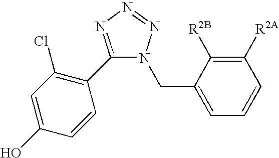 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-17) |
| 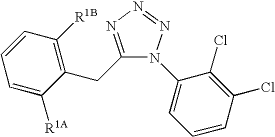 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-18) |
| 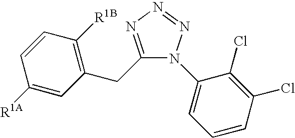 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-19) |
| 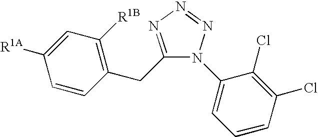 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-20) |
| 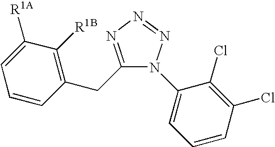 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-21) |
| 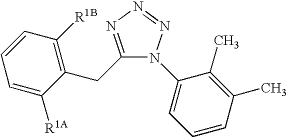 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-22) |
| 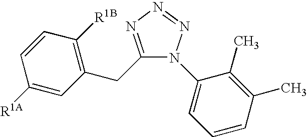 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-23) |
| 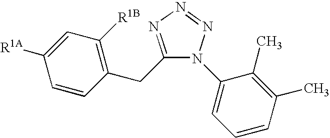 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-24) |
| 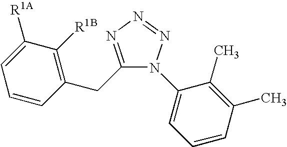 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-25) |
| 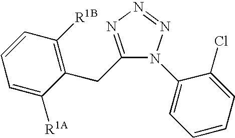 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-26) |
| 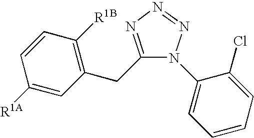 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-27) |
| 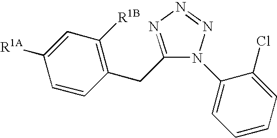 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-28) |
| 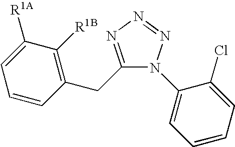 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-29) |
| 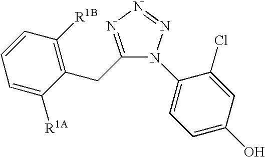 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-a-30) |
| 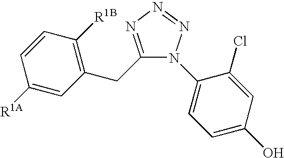 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-31) |
| 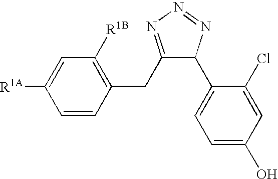 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-a-32) |
| 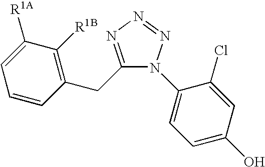 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-1) |
| 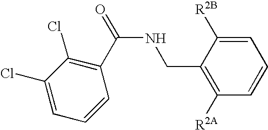 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-2) |
| 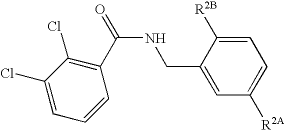 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-3) |
| 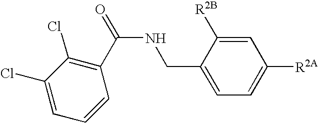 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-4) |
| 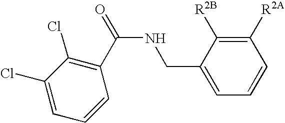 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-5) |
| 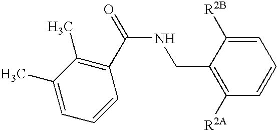 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-6) |
| 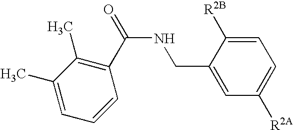 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-7) |
| 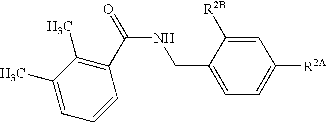 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-8) |
| 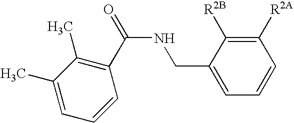 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-9) |
| 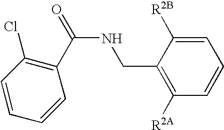 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-10) |
| 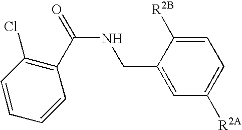 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-11) |
| 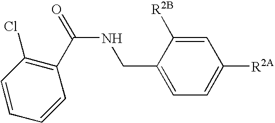 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-12) |
| 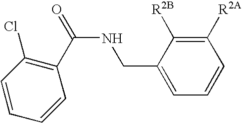 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-13) |
| 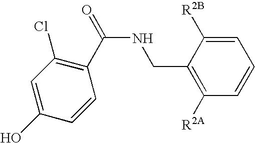 | |

| | R^{2A} | R^{2B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-14) |
| 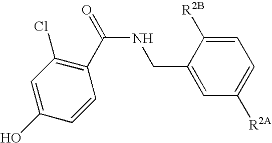 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-15) |
| 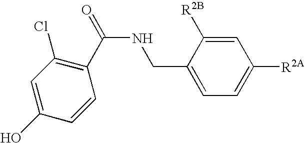 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-16) |
| 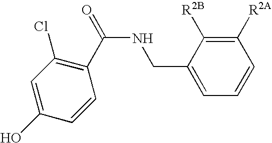 | |

| | R^{2A} | R^{2B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-17) |
| 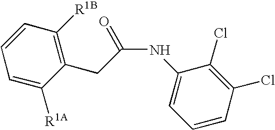 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-18) |
| 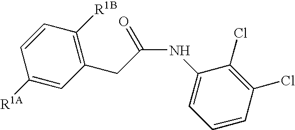 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-19) |
| 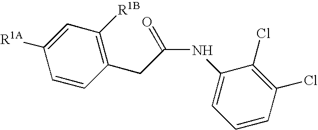 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-20) |
| 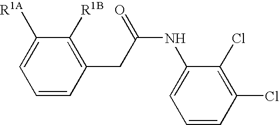 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-21) |
| 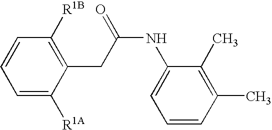 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-22) |
| 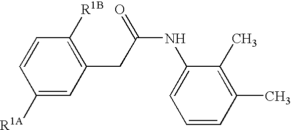 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-23) |
| 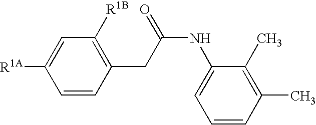 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | HBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-24) |
| 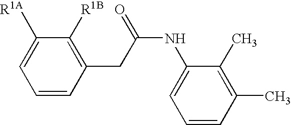 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-25) |
| 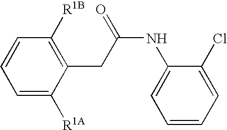 | |

| | |
|---|---|
| R^{1A} | R^{1B} |

| | | |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-26) |
| 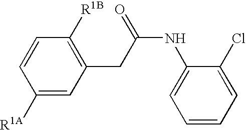 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-27) |
| 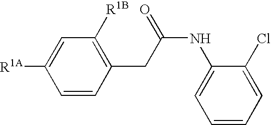 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-28) |
| 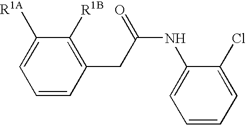 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-29) |
| 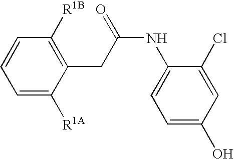 | |

| | R^{1A} | R^{1B} |
|---|---|---|
| 1 | H | H |
| 2 | H | CH₃ |
| 3 | H | Cl |
| 4 | H | F |
| 5 | H | CF₃ |
| 6 | H | Ph |
| 7 | H | OH |
| 8 | H | OCH₃ |
| 9 | H | OiPr |
| 10 | H | OPh |
| 11 | H | CH₂Ph |
| 12 | H | CH₂CH₂Ph |
| 13 | H | OBzl |
| 14 | H | NHBzl |
| 15 | H | NCH₃Bzl |
| 16 | H | NH₂ |
| 17 | H | NHCH₃ |
| 18 | H | N(CH₃)₂ |
| 19 | CH₃ | CH₃ |
| 20 | CH₃ | Cl |
| 21 | CH₃ | F |
| 22 | CH₃ | CF₃ |
| 23 | CH₃ | Ph |
| 24 | CH₃ | OH |
| 25 | CH₃ | OCH₃ |
| 26 | CH₃ | OiPr |
| 27 | CH₃ | OPh |
| 28 | CH₃ | CH₂Ph |
| 29 | CH₃ | CH₂CH₂Ph |
| 30 | CH₃ | OBzl |
| 31 | CH₃ | NHBzl |
| 32 | CH₃ | NCH₃Bzl |
| 33 | CH₃ | NH₂ |
| 34 | CH₃ | NHCH₃ |
| 35 | CH₃ | N(CH₃)₂ |
| 36 | F | Cl |
| 37 | F | F |
| 39 | F | CF₃ |
| 40 | F | Ph |
| 41 | F | OH |
| 42 | F | OCH₃ |
| 43 | F | OiPr |
| 44 | F | OPh |
| 45 | F | CH₂Ph |
| 46 | F | CH₂CH₂Ph |
| 47 | F | OBzl |
| 48 | F | NHBzl |
| 49 | F | NCH₃Bzl |
| 50 | F | NH₂ |
| 51 | F | NHCH₃ |
| 52 | F | N(CH₃)₂ |
| 53 | Cl | Cl |
| 54 | Cl | CF₃ |
| 55 | Cl | Ph |
| 56 | Cl | OH |
| 57 | Cl | OCH₃ |
| 58 | Cl | OiPr |
| 59 | Cl | OPh |
| 60 | Cl | CH₂Ph |
| 61 | Cl | CH₂CH₂Ph |
| 62 | Cl | OBzl |
| 63 | Cl | NHBzl |
| 64 | Cl | NCH₃Bzl |
| 65 | Cl | NH₂ |
| 66 | Cl | NHCH₃ |
| 67 | Cl | N(CH₃)₂ |
| 68 | N(CH₃)₂ | CF₃ |
| 69 | N(CH₃)₂ | Ph |
| 70 | N(CH₃)₂ | OH |
| 71 | N(CH₃)₂ | OCH₃ |
| 72 | N(CH₃)₂ | OiPr |
| 73 | N(CH₃)₂ | OPh |
| 74 | N(CH₃)₂ | CH₂Ph |
| 75 | N(CH₃)₂ | CH₂CH₂Ph |
| 76 | N(CH₃)₂ | OBzl |
| 77 | N(CH₃)₂ | NHBzl |
| 78 | N(CH₃)₂ | NCH₃Bzl |
| 79 | N(CH₃)₂ | NH₂ |
| 80 | N(CH₃)₂ | NHCH₃ |
| 81 | N(CH₃)₂ | N(CH₃)₂ |

| | |
|---|---|
| | (I-b-30) |
| 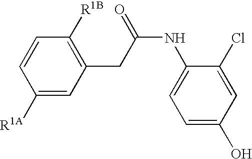 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-31) |
| 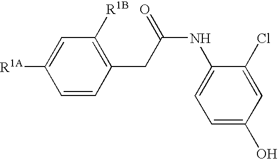 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

| | |
|---|---|
| | (I-b-32) |
| 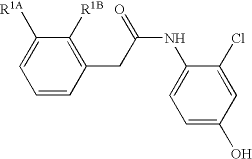 | |

| | R^{1A} | R^{1B} |
|---|---|---|
| 1 | CH₃ | H |
| 2 | CH₃ | CH₃ |
| 3 | CH₃ | Cl |
| 4 | CH₃ | F |
| 5 | CH₃ | CF₃ |
| 6 | CH₃ | Ph |
| 7 | CH₃ | OH |
| 8 | CH₃ | OCH₃ |
| 9 | CH₃ | OiPr |
| 10 | CH₃ | OPh |
| 11 | CH₃ | CH₂Ph |
| 12 | CH₃ | CH₂CH₂Ph |
| 13 | CH₃ | OBzl |
| 14 | CH₃ | NHBzl |
| 15 | CH₃ | NCH₃Bzl |
| 16 | CH₃ | NH₂ |
| 17 | CH₃ | NHCH₃ |
| 18 | CH₃ | N(CH₃)₂ |
| 19 | F | H |
| 20 | F | CH₃ |
| 21 | F | Cl |
| 22 | F | F |
| 23 | F | CF₃ |
| 24 | F | Ph |
| 25 | F | OH |
| 26 | F | OCH₃ |
| 27 | F | OiPr |
| 28 | F | OPh |
| 29 | F | CH₂Ph |
| 30 | F | CH₂CH₂Ph |
| 31 | F | OBzl |
| 32 | F | NHBzl |
| 33 | F | NCH₃Bzl |
| 34 | F | NH₂ |
| 35 | F | NHCH₃ |
| 36 | F | N(CH₃)₂ |
| 37 | Cl | H |
| 38 | Cl | CH₃ |
| 39 | Cl | Cl |
| 40 | Cl | F |
| 41 | Cl | CF₃ |
| 42 | Cl | Ph |
| 43 | Cl | OH |
| 44 | Cl | OCH₃ |
| 45 | Cl | OiPr |
| 46 | Cl | OPh |
| 47 | Cl | CH₂Ph |
| 48 | Cl | CH₂CH₂Ph |
| 49 | Cl | OBzl |
| 50 | Cl | NHBzl |
| 51 | Cl | NCH₃Bzl |
| 52 | Cl | NH₂ |
| 53 | Cl | NHCH₃ |
| 54 | Cl | N(CH₃)₂ |
| 55 | N(CH₃)₂ | H |
| 56 | N(CH₃)₂ | CH₃ |
| 57 | N(CH₃)₂ | Cl |
| 58 | N(CH₃)₂ | F |
| 59 | N(CH₃)₂ | CF₃ |
| 60 | N(CH₃)₂ | Ph |
| 61 | N(CH₃)₂ | OH |
| 62 | N(CH₃)₂ | OCH₃ |
| 63 | N(CH₃)₂ | OiPr |
| 64 | N(CH₃)₂ | OPh |
| 65 | N(CH₃)₂ | CH₂Ph |
| 66 | N(CH₃)₂ | CH₂CH₂Ph |
| 67 | N(CH₃)₂ | OBzl |
| 68 | N(CH₃)₂ | NHBzl |
| 69 | N(CH₃)₂ | NCH₃Bzl |
| 70 | N(CH₃)₂ | NH₂ |
| 71 | N(CH₃)₂ | NHCH₃ |
| 72 | N(CH₃)₂ | N(CH₃)₂ |

Quite preferably, the compounds of formulas (I)-(V) are as follows:

| | |
|---|---|
| Compound 1(16) | 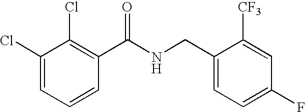 |
| Compound 1(110) | 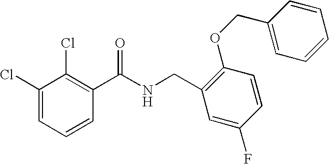 |
| Compound 2(14) | 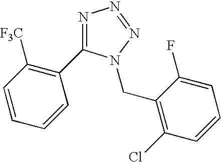 |
| Compound 2(71) | 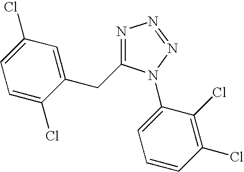 |
| Compound 2(74) | 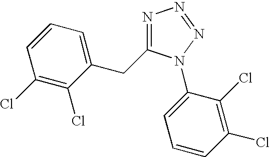 |
| Compound 2(105) | 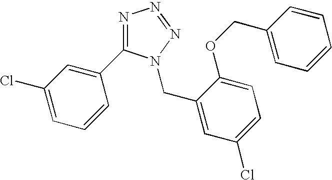 |

The compounds of formula (VI) that are preferred according to the invention are selected from the compounds of the following formulas (VI-a), (VI-b), (VI-c), (VI-d), (VI-e), (VI-f), (VI-g), (VI-h), (VI-i), (VI-j), (VI-k), (VI-m), in which the symbols have the meanings given previously:

| | |
|---|---|
| 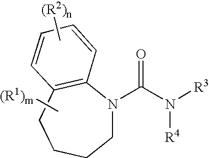 | (VI-a) |
| 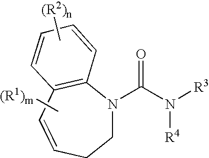 | (VI-b) |
| 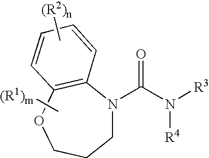 | (VI-c) |
| 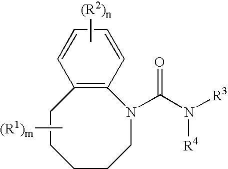 | (VI-d) |
| 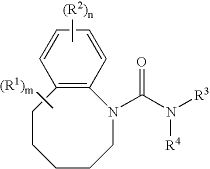 | (VI-e) |
| 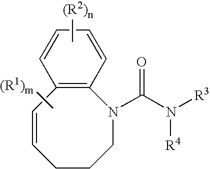 | (VI-f) |
| 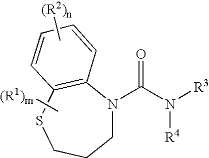 | (VI-g) |
| 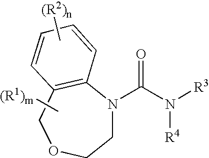 | (VI-h) |
| 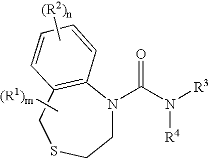 | (VI-j) |
| 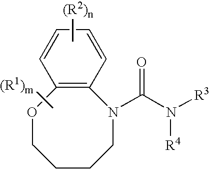 | (VI-k) |
| 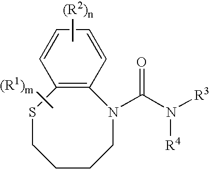 | (VI-m) |

Advantageously, the compounds of formula (VI) are selected from the compounds of the following formulas (VI-a-1), (VI-a-2), (VI-a-3), (VI-a-4), (VI-a-5), (VI-a-6), (VI-a-7), (VI-a-8), (VI-a-9), (VI-a-10), (VI-a-11), (VI-a-12), (VI-a-13):

| | |
|---|---|
| | (VI-a-1) |
| 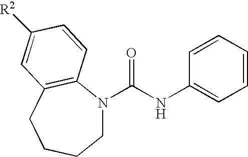 | |

| | R² |
|---|---|
| 1 | H |
| 2 | CH₃ |
| 3 | OH |
| 4 | OCH₃ |
| 5 | NH₂ |
| 6 | NHCH₃ |
| 7 | N(CH₃)₂ |
| 8 | NO₂ |
| 9 | F |
| 10 | Cl |

| | |
|---|---|
| | (VI-a-2) |
| 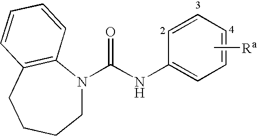 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |

| | | | | | | | | (VI-a-3) |
|---|---|---|---|---|---|---|---|---|
| 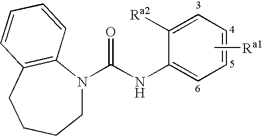 | | | | | | | | |
| | R^{a1} | R^{a2} | | R^{a1} | R^{a2} | | R^{a1} | R^{a2} |
| 1-1 | H | OH | 2-1 | H | NH₂ | 3-1 | H | NHCH₃ |
| 1-2 | 3-CH₃ | OH | 2-2 | 3-CH₃ | NH₂ | 3-2 | 3-CH₃ | NHCH₃ |
| 1-3 | 3-OCH₃ | OH | 2-3 | 3-OCH₃ | NH₂ | 3-3 | 3-OCH₃ | NHCH₃ |
| 1-4 | 3-NO₂ | OH | 2-4 | 3-NO₂ | NH₂ | 3-4 | 3-NO₂ | NHCH₃ |
| 1-5 | 3-F | OH | 2-5 | 3-F | NH₂ | 3-5 | 3-F | NHCH₃ |
| 1-6 | 3-Cl | OH | 2-6 | 3-Cl | NH₂ | 3-6 | 3-Cl | NHCH₃ |
| 1-7 | 4-CH₃ | OH | 2-7 | 4-CH₃ | NH₂ | 3-7 | 4-CH₃ | NHCH₃ |
| 1-8 | 4-OCH₃ | OH | 2-8 | 4-OCH₃ | NH₂ | 3-8 | 4-OCH₃ | NHCH₃ |
| 1-9 | 4-NO₂ | OH | 2-9 | 4-NO₂ | NH₂ | 3-9 | 4-NO₂ | NHCH₃ |
| 1-10 | 4-F | OH | 2-10 | 4-F | NH₂ | 3-10 | 4-F | NHCH₃ |
| 1-11 | 4-Cl | OH | 2-11 | 4-Cl | NH₂ | 3-11 | 4-Cl | NHCH₃ |
| 1-12 | 5-CH₃ | OH | 2-12 | 5-CH₃ | NH₂ | 3-12 | 5-CH₃ | NHCH₃ |
| 1-13 | 5-OCH₃ | OH | 2-13 | 5-OCH₃ | NH₂ | 3-13 | 5-OCH₃ | NHCH₃ |
| 1-14 | 5-NO₂ | OH | 2-14 | 5-NO₂ | NH₂ | 3-14 | 5-NO₂ | NHCH₃ |
| 1-15 | 5-F | OH | 2-15 | 5-F | NH₂ | 3-15 | 5-F | NHCH₃ |
| 1-16 | 5-Cl | OH | 2-16 | 5-Cl | NH₂ | 3-16 | 5-Cl | NHCH₃ |
| 1-17 | 6-CH₃ | OH | 2-17 | 6-CH₃ | NH₂ | 3-17 | 6-CH₃ | NHCH₃ |
| 1-18 | 6-OCH₃ | OH | 2-18 | 6-OCH₃ | NH₂ | 3-18 | 6-OCH₃ | NHCH₃ |
| 1-19 | 6-NO₂ | OH | 2-19 | 6-NO₂ | NH₂ | 3-19 | 6-NO₂ | NHCH₃ |
| 1-20 | 6-F | OH | 2-20 | 6-F | NH₂ | 3-20 | 6-F | NHCH₃ |
| 1-21 | 6-Cl | OH | 2-21 | 6-Cl | NH₂ | 3-21 | 6-Cl | NHCH₃ |

| | | | | | | | | (VI-a-4) |
|---|---|---|---|---|---|---|---|---|
| 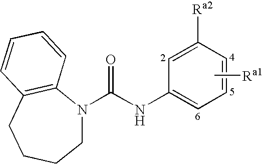 | | | | | | | | |
| | R^{a1} | R^{a2} | | R^{a1} | R^{a2} | | R^{a1} | R^{a2} |
| 1-1 | H | OH | 2-1 | H | NH₂ | 3-1 | H | NHCH₃ |
| 1-2 | 2-CH₃ | OH | 2-2 | 2-CH₃ | NH₂ | 3-2 | 2-CH₃ | NHCH₃ |
| 1-3 | 2-OCH₃ | OH | 2-3 | 2-OCH₃ | NH₂ | 3-3 | 2-OCH₃ | NHCH₃ |
| 1-4 | 2-NO₂ | OH | 2-4 | 2-NO₂ | NH₂ | 3-4 | 2-NO₂ | NHCH₃ |
| 1-5 | 2-F | OH | 2-5 | 2-F | NH₂ | 3-5 | 2-F | NHCH₃ |
| 1-6 | 2-Cl | OH | 2-6 | 2-Cl | NH₂ | 3-6 | 2-Cl | NHCH₃ |
| 1-7 | 4-CH₃ | OH | 2-7 | 4-CH₃ | NH₂ | 3-7 | 4-CH₃ | NHCH₃ |
| 1-8 | 4-OCH₃ | OH | 2-8 | 4-OCH₃ | NH₂ | 3-8 | 4-OCH₃ | NHCH₃ |
| 1-9 | 4-NO₂ | OH | 2-9 | 4-NO₂ | NH₂ | 3-9 | 4-NO₂ | NHCH₃ |
| 1-10 | 4-F | OH | 2-10 | 4-F | NH₂ | 3-10 | 4-F | NHCH₃ |
| 1-11 | 4-Cl | OH | 2-11 | 4-Cl | NH₂ | 3-11 | 4-Cl | NHCH₃ |
| 1-12 | 5-CH₃ | OH | 2-12 | 5-CH₃ | NH₂ | 3-12 | 5-CH₃ | NHCH₃ |
| 1-13 | 5-OCH₃ | OH | 2-13 | 5-OCH₃ | NH₂ | 3-13 | 5-OCH₃ | NHCH₃ |
| 1-14 | 5-NO₂ | OH | 2-14 | 5-NO₂ | NH₂ | 3-14 | 5-NO₂ | NHCH₃ |
| 1-15 | 5-F | OH | 2-15 | 5-F | NH₂ | 3-15 | 5-F | NHCH₃ |
| 1-16 | 5-Cl | OH | 2-16 | 5-Cl | NH₂ | 3-16 | 5-Cl | NHCH₃ |
| 1-17 | 6-CH₃ | OH | 2-17 | 6-CH₃ | NH₂ | 3-17 | 6-CH₃ | NHCH₃ |
| 1-18 | 6-OCH₃ | OH | 2-18 | 6-OCH₃ | NH₂ | 3-18 | 6-OCH₃ | NHCH₃ |
| 1-19 | 6-NO₂ | OH | 2-19 | 6-NO₂ | NH₂ | 3-19 | 6-NO₂ | NHCH₃ |
| 1-20 | 6-F | OH | 2-20 | 6-F | NH₂ | 3-20 | 6-F | NHCH₃ |
| 1-21 | 6-Cl | OH | 2-21 | 6-Cl | NH₂ | 3-21 | 6-Cl | NHCH₃ |

| | | | | | | | | (VI-a-5) |
|---|---|---|---|---|---|---|---|---|
| 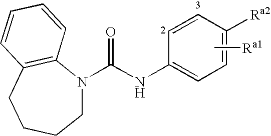 | | | | | | | | |
| | R^{a1} | R^{a2} | | R^{a1} | R^{a2} | | R^{a1} | R^{a2} |
| 1-1 | H | OH | 2-1 | H | NH₂ | 3-1 | H | NHCH₃ |
| 1-2 | 2-CH₃ | OH | 2-2 | 2-CH₃ | NH₂ | 3-2 | 2-CH₃ | NHCH₃ |
| 1-3 | 2-OCH₃ | OH | 2-3 | 2-OCH₃ | NH₂ | 3-3 | 2-OCH₃ | NHCH₃ |
| 1-4 | 2-NO₂ | OH | 2-4 | 2-NO₂ | NH₂ | 3-4 | 2-NO₂ | NHCH₃ |
| 1-5 | 2-F | OH | 2-5 | 2-F | NH₂ | 3-5 | 2-F | NHCH₃ |
| 1-6 | 2-Cl | OH | 2-6 | 2-Cl | NH₂ | 3-6 | 2-Cl | NHCH₃ |
| 1-7 | 3-CH₃ | OH | 2-7 | 3-CH₃ | NH₂ | 3-7 | 3-CH₃ | NHCH₃ |
| 1-8 | 3-OCH₃ | OH | 2-8 | 3-OCH₃ | NH₂ | 3-8 | 3-OCH₃ | NHCH₃ |
| 1-9 | 3-NO₂ | OH | 2-9 | 3-NO₂ | NH₂ | 3-9 | 3-NO₂ | NHCH₃ |
| 1-10 | 3-F | OH | 2-10 | 3-F | NH₂ | 3-10 | 3-F | NHCH₃ |
| 1-11 | 3-Cl | OH | 2-11 | 3-Cl | NH₂ | 3-11 | 3-Cl | NHCH₃ |

| | |
|---|---|
| | (VI-a-6) |
| 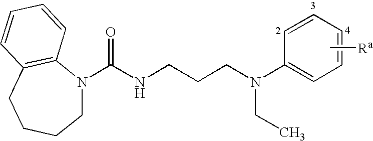 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |
| 17 | 2-NH₂ |
| 18 | 2-CN |
| 19 | 2-NHCH₃ |
| 20 | 2-COOH |
| 21 | 2-OH |
| 22 | 3-NH₂ |
| 23 | 3-CN |
| 24 | 3-NHCH₃ |
| 25 | 3-COOH |
| 26 | 3-OH |
| 27 | 4-NH₂ |
| 28 | 4-CN |
| 29 | 4-NHCH₃ |
| 30 | 4-COOH |
| 31 | 4-OH |
| 32 | 4-COOCH₃ |

| | |
|---|---|
| | (VI-a-7) |
| 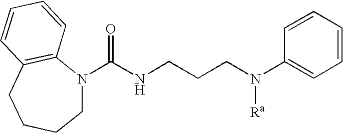 | |

| | R^{a} |
|---|---|
| 1 | H |
| 2 | CH₃ |
| 3 | CH₂CH₃ |
| 4 | CH(CH₃)₂ |
| 5 | Ph |
| 6 | CH₂Ph |

| | |
|---|---|
| | (VI-a-8) |
| 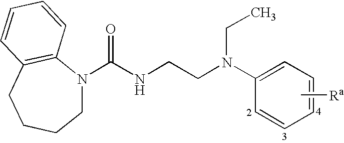 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |
| 17 | 2-NH₂ |
| 18 | 2-CN |
| 19 | 2-NHCH₃ |
| 20 | 2-COOH |
| 21 | 2-OH |
| 22 | 3-NH₂ |
| 23 | 3-CN |
| 24 | 3-NHCH₃ |
| 25 | 3-COOH |
| 26 | 3-OH |
| 27 | 4-NH₂ |
| 28 | 4-CN |
| 29 | 4-NHCH₃ |
| 30 | 4-COOH |
| 31 | 4-OH |
| 32 | 4-COOCH₃ |

| | |
|---|---|
| | (VI-a-9) |
| 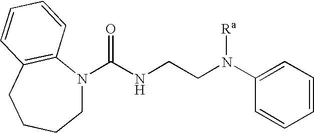 | |

| | R^{a} |
|---|---|
| 1 | H |
| 2 | CH₃ |
| 3 | CH₂CH₃ |
| 4 | CH(CH₃)₂ |
| 5 | Ph |
| 6 | CH₂Ph |

| | |
|---|---|
| | (VI-a-10) |
| 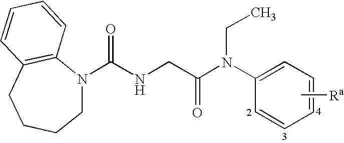 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |
| 17 | 2-NH₂ |
| 18 | 2-CN |
| 19 | 2-NHCH₃ |
| 20 | 2-COOH |
| 21 | 2-OH |
| 22 | 3-NH₂ |
| 23 | 3-CN |
| 24 | 3-NHCH₃ |
| 25 | 3-COOH |
| 26 | 3-OH |
| 27 | 4-NH₂ |
| 28 | 4-CN |
| 29 | 4-NHCH₃ |
| 30 | 4-COOH |
| 31 | 4-OH |
| 32 | 4-COOCH₃ |

| | |
|---|---|
| | (VI-a-11) |
| 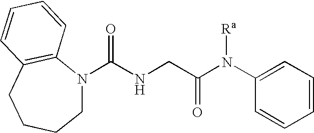 | |

| | R^{a} |
|---|---|
| 1 | H |
| 2 | CH₃ |
| 3 | CH₂CH₃ |
| 4 | CH(CH₃)₂ |
| 5 | Ph |
| 6 | CH₂Ph |

| | |
|---|---|
| | (VI-a-12) |
| 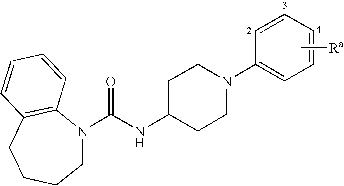 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |
| 17 | 2-NH₂ |
| 18 | 2-CN |
| 19 | 2-NHCH₃ |
| 20 | 2-COOH |
| 21 | 2-OH |
| 22 | 3-NH₂ |
| 23 | 3-CN |
| 24 | 3-NHCH₃ |
| 25 | 3-COOH |
| 26 | 3-OH |
| 27 | 4-NH₂ |
| 28 | 4-CN |
| 29 | 4-NHCH₃ |
| 30 | 4-COOH |
| 31 | 4-OH |
| 32 | 4-COOCH₃ |

| | |
|---|---|
| | (VI-a-13) |
| 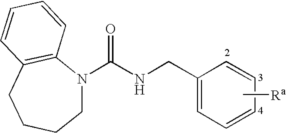 | |

| | R^{a} |
|---|---|
| 1 | H |
| 2 | 2-CH₃ |
| 3 | 2-OCH₃ |
| 4 | 2-NO₂ |
| 5 | 2-F |
| 6 | 2-Cl |
| 7 | 3-CH₃ |
| 8 | 3-OCH₃ |
| 9 | 3-NO₂ |
| 10 | 3-F |
| 11 | 3-Cl |
| 12 | 4-CH₃ |
| 13 | 4-OCH₃ |
| 14 | 4-NO₂ |
| 15 | 4-F |
| 16 | 4-Cl |
| 17 | 2-NH₂ |
| 18 | 2-CN |
| 19 | 2-NHCH₃ |
| 20 | 2-COOH |
| 21 | 2-OH |
| 22 | 3-NH₂ |
| 23 | 3-CN |
| 24 | 3-NHCH₃ |
| 25 | 3-COOH |
| 26 | 3-OH |
| 27 | 4-NH₂ |
| 28 | 4-CN |
| 29 | 4-NHCH₃ |
| 30 | 4-COOH |
| 31 | 4-OH |
| 32 | 4-COOCH₃ |

Quite preferably, the compounds of formula (VI) are selected from the following compounds:
8-fluoro-1-phenylcarbamoyl-2,3,4,5-tetrahydro-1H-1-benzazepine;
1-(2,6-dichlorophenylcarbamoyl)-2,3,4,5-tetrahydro-1H-1-benzazepine;
1-(4-hydroxyphenylcarbamoyl)-2,3,4,5-tetrahydro-1H-1-benzazepine.

The present invention further relates to the use of at least one PBR antagonist as defined previously, for the preparation of a composition, notably a cosmetic or dermatological composition, intended for treating disorders connected with problems of keratinization, in particular connected with excessive differentiation of keratinocytes, or with disorders of synthesis of the constituents of the extracellular matrix of the epidermis.

The quantity of antagonist of the peripheral benzodiazepine receptors (PBR) that can be used according to the invention depends of course on the desired effect and can therefore vary widely. Generally, the PBR antagonist will be present in an amount that is sufficient to give a significant increase in the production of epidermal glycosaminoglycans and advantageously to increase by at least 10% the production of glycosaminoglycans by a culture of keratinocytes. Moreover, the PBR antagonist can be present in an amount that gives a significant reduction in the differentiation of the keratinocytes and notably in an amount that provides, at the level of the epidermal cells, a concentration that is sufficient to reduce the activity of TGK1 of a culture of human keratinocytes by at least 10%.

To give an order of magnitude, the antagonist of the peripheral benzodiazepine receptors (PBR) can be used in an amount representing from 0.001% to 10% of the total weight of the composition, preferably in an amount representing from 0.01% to 5%, and more preferably from 0.05 to 1% of the total weight of the composition.

The compositions that can be used for application of the invention can be in a form that is suitable for the various routes of administration, notably for administration by the oral or topical route or by injection.

For use by the oral route, the composition can notably be in the form of hard or soft capsules, coated tablets, granules, tablets, chewable pastilles, gels or oral syrups or any other form known by a person skilled in the art.

The composition is more particularly suitable for topical application on keratinous materials, notably on the skin, the mucosae and/or the integumentary appendages.

This composition can be more or less fluid and have the form of a white or colored cream, an ointment, a milk, a lotion, a serum, a paste, a mousse. It can also be in solid form, in particular in the form of a stick. It can be used as a skin care product and/or as a skin make-up product.

The composition according to the invention can be in all the galenical forms normally used in the field of cosmetics, and it can notably be in the form of an oily solution, optionally gelled, of a dispersion of the lotion type, optionally two-phase, of an emulsion obtained by dispersion of an oil phase in an aqueous phase (O/W) or vice-versa (W/O), or of a triple emulsion (W/O/W or O/W/O) or multiple, or of a vesicular dispersion of ionic and/or nonionic type. These compositions are prepared by the usual methods.

When the composition used according to the invention is an emulsion, the proportion of the oily phase can range from 5 to 80 wt. %, and preferably from 5 to 50 wt. % relative to the total weight of the composition. The oils, the emulsifiers and the co-emulsifiers used in the composition in the form of emulsion are selected from those used conventionally in the field in question. The emulsifier and the co-emulsifier are present, in the composition, in a proportion ranging from 0.3 to 30 wt. %, and preferably from 0.5 to 20 wt. % relative to the total weight of the composition.

The following may be mentioned as oils that can be used in the invention: hydrocarbons of mineral origin (mineral oil) or synthetic (liquid paraffin, isohexadecane), oils of vegetable origin (almond oil, apricot oil, liquid fraction of shea butter, avocado oil, soy oil), oils of animal origin (lanolin), synthetic oils (perhydrosqualene, pentaerythrityl tetraoctanoate), silicone oils (cyclopentasiloxane and cyclohexasiloxane) and fluorinated oils (perfluoropolyethers). The following may also be used as fatty substances: fatty alcohols (cetyl or stearyl alcohol), fatty acids (stearic acid), waxes (carnauba wax, ozokerite, beeswax).

As emulsifiers and co-emulsifiers for use in the invention, we may mention for example the esters of fatty acid and of polyethylene glycol such as the stearate of PEG-100 and the stearate of PEG-20 and the esters of fatty acid and of glycerol such as glyceryl stearate.

In a known manner, the composition used according to the invention can also contain the additives that are usually employed in the field of cosmetics, such as hydrophilic or lipophilic gelling agents, hydrophilic or lipophilic actives, preservatives, antioxidants, solvents, perfumes, fillers, filters, pigments, odor absorbers and coloring matter. The amounts of these various additives are those conventionally used in the field in question, and for example from 0.01 to 20% of the total weight of the composition. These additives, according to their nature, can be added to the oily phase, to the aqueous phase or to the lipid vesicles. In any event, these additives, as well as their proportions, will be chosen so as not to have an adverse effect on the anti-age properties of the PBR antagonists.

As hydrophilic gelling agents, we may mention in particular carboxyvinyl polymers (carbomers), acrylic copolymers such as acrylate/alkylacrylate copolymers, polyacrylamides, polysaccharides, natural gums and clays, and, as lipophilic gelling agents, we may mention modified clays such as bentones, metal salts of fatty acids, hydrophobic silica and polyethylenes.

As fillers, we may mention, for example, polyamide (Nylon) particles of spherical shape or in the form of microfibers; poly(methyl methacrylate) microspheres; powders of ethylene-acrylate copolymer; expanded powders such as hollow microspheres and, notably, microspheres formed from a terpolymer of vinylidene chloride, acrylonitrile and methacrylate and marketed under the designation EXPANCEL by the company Kemanord Plast; powders of natural organic materials such as starch powders, notably maize, wheat or rice starch, crosslinked or not, such as powdered starch crosslinked by octenylsuccinate anhydride; silicone resin microbeads such as those marketed under the designation TOSPEARL by the company Toshiba Silicone; silica; metal oxides such as titanium dioxide or zinc oxide; mica; and mixtures thereof.

According to one embodiment of the invention, the compositions that can be used additionally contain at least one active selected from hydrating agents, depigmenting agents, antiglycation agents, NO synthase inhibitors, agents that stimulate the synthesis of dermal or epidermal macromolecules and/or prevent their degradation, agents that stimulate the proliferation of fibroblasts or keratinocytes, muscle relaxants or dermal relaxants, lifting agents, antipollution or antiradical agents, soothing agents and agents acting on the energy metabolism of the cells.

The concentration of these various additional actives will be adapted by a person skilled in the art in relation to the desired effect, but will generally vary from 0.001% to 20%, notably from 0.01% to 10%, relative to the total weight of the composition.

By “hydrating agent”, we mean:
either a compound acting on the barrier function, for the purpose of maintaining the hydration of the stratum corneum, or an occlusive compound. We may mention ceramides, sphingoid-based compounds, lecithins, glycosphingolipids, phospholipids, cholesterol and derivatives thereof, phytosterols (stigmasterol, β-sitosterol, campesterol), essential fatty acids, 1-2 diacylglycerol, 4-chromanone, pentacyclic triterpenes such as ursolic acid, petroleum jelly and lanolin;
or a compound directly increasing the water content of the stratum corneum, such as trehalose and derivatives thereof, hyaluronic acid and derivatives thereof, glycerol, pentanediol, sodium pidolate, serine, xylitol, sodium lactate, glycerol polyacrylate, ectoin and derivatives thereof, chitosan, oligo- and polysaccharides, cyclic carbonates, N-lauroyl pyrrolidone carboxylic acid, and N-α-benzoyl-L-arginine;
or a compound that activates the sebaceous glands such as steroid derivatives (including DHEA, its 7-oxide and 17-alkyl derivatives and sapogenins), methyl dihydrojasmonate, and vitamin D and derivatives thereof.

These compounds can represent from 0.001% to 30%, and preferably from 0.01 to 20%, of the total weight of the composition according to the invention.

The depigmenting or anti-pigmenting agents that can be included in the composition according to the present invention comprise, for example, the following compounds: kojic acid; ellagic acid; arbutin and derivatives thereof such as those described in applications EP-895 779 and EP-524 109; hydroquinone; derivatives of aminophenol such as those described in applications WO 99/10318 and WO 99/32077, and in particular N-cholesteryloxycarbonyl-para-aminophenol and N-ethyloxycarbonyl-para-aminophenol; iminophenol derivatives, in particular those described in application WO 99/22707; L-2-oxothiazolidine-4-carboxylic acid or procysteine, as well as its salts and esters; calcium D-pantetheine sulfonate, ascorbic acid and derivatives thereof, notably ascorbyl glycoside; and extracts from plants, in particular licorice, mulberry, skullcap and Bacopa monnieri, without this list being limitative.

“Antiglycation agent” means a compound that prevents and/or decreases the glycation of the proteins of the skin, in particular the proteins of the dermis such as collagen. Examples of antiglycation agents are vegetable extracts of the Ericaceae family, such as extract of bilberry (Vaccinium angustifolium); ergothioneine and derivatives thereof; and the hydroxystilbenes and their derivatives, such as resveratrol and 3,3′,5,5′-tetrahydroxystilbene. These antiglycation agents are described in applications FR 2 802 425, FR 2 810 548, FR 2 796 278 and FR 2 802 420, respectively.

Examples of inhibitors of NO-synthase suitable for use in the present invention comprise notably a vegetable extract of the species Vitis vinifera which is notably marketed by the company Euromed under the designation “Leucocyanidines de raisins extra”, or by the company Indena under the designation Leucoselect®, or finally by the company Hansen under the designation “Extrait de marc de raisin”; a vegetable extract of the species Olea europaea which is preferably obtained from olive leaves and is notably marketed by the company VINYALS as a dry extract, or by the company Biologia & Technologia under the trade name Eurol BT; and an extract from a plant of the species Ginkgo biloba which is preferably a dry aqueous extract of this plant sold by the company Beaufour under the trade name “Ginkgo biloba extrait standard”.

The agents promoting synthesis of the constituents of the extracellular matrix are notably agents that stimulate the synthesis of the macromolecules of the dermis or of the epidermis or prevent their degradation. Among the actives stimulating the macromolecules of the dermis or preventing their degradation, we may mention those which act:
either on the synthesis of collagen, such as extracts of Centella asiatica; asiaticosides and derivatives; ascorbic acid or vitamin C and derivatives thereof; synthetic peptides such as iamin, biopeptide CL or palmitoyl oligopeptide marketed by the company SEDERMA; peptides extracted from plants, such as soy hydrolyzate marketed by the company COLETICA under the trade name Phytokine®; and plant hormones such as auxins and lignans.
or on the synthesis of elastin, such as the extract of Saccharomyces cerivisiae marketed by the company LSN under the trade name Cytovitin®; the extract of the alga Macrocystis pyrifera marketed by the company SECMA under the trade name Kelpadelie®,
or on the synthesis of the glycosaminoglycans, such as the product from the fermentation of milk by Lactobacillus vulgaris, marketed by the company BROOKS under the trade name Biomin yogourth®; the extract of brown alga Padina pavonica marketed by the company ALBAN MULLER under the trade name HSP3®; the extract of Saccharomyces cerevisiae obtainable notably from the company SILAB under the trade name Firmalift® or from the company LSN under the trade name Cytovitin®; derivatives of xylose such as Aquaxyl®; and C-glycoside derivatives such as those described in application EP1345919, in particular C-β-D-xylopyranoside-2-hydroxypropane and its physiologically acceptable salts;
or on the synthesis of fibronectin, such as the extract of zooplankton Salina marketed by the company SEPORGA under the trade name GP4G®; yeast extract available notably from the company ALBAN MULLER under the trade name Drieline®; and palmitoyl pentapeptide marketed by the company SEDERMA under the trade name Matrixil®;
or on the inhibition of metalloproteinases (MMP) such as, more particularly, MMP 1, 2, 3, 9. We may mention: retinoids and derivatives, oligopeptides and lipopeptides, lipo-amino acids, malt extract marketed by the company COLETICA under the trade name Collalift®; extracts of bilberry or of rosemary; lycopene; isoflavones, their derivatives or plant extracts containing them, in particular soy extracts (marketed for example by the company ICHIMARU PHARCOS under the trade name Flavosterone SB®), of red clover, of flax, of kakkon or of sage;
or on the inhibition of the serine proteases such as leukocytic elastase or cathepsin G. We may mention: peptide extract of seeds of Leguminose (Pisum sativum) marketed by the company LSN under the trade name Parelastyl®; heparinoids; pseudodipeptides such as {2-[acetyl-(3-trifluoromethyl-phenyl)-amino]-3-methyl-butyrylamino}acetic acid; and inhibitors of elastase such as the N-acyl-aminoamides described in application EP 1 292 608, in particular {2-[acetyl-(3-trifluoromethyl-phenyl)-amino]-3-methyl-butyrylamino}acetic acid, {2-[acetyl-(3-trifluoromethyl-phenyl)-amino]-3-methyl-butyrylamino}ethyl acetate, [2-(acetyl-benzyl-amino)-3-methyl-butyrylamino]acetic acid, [2-(acetyl-benzyl-amino)-3-methyl-butyrylamino]ethyl acetate, (2-{benzyl-[(diethoxy-phosphoryl)-acetyl]-amino}-3-methyl-butyrylamino) ethyl acetate.

Among the actives that stimulate the epidermal macromolecules, such as fillagrin and the keratins, we may notably mention the lupin extract marketed by the company SILAB under the trade name Structurine®; the extract from buds of the beech Fagus sylvatica marketed by the company GATTEFOSSE under the trade name Gatuline®; and the extract of zooplankton Salina marketed by the company SEPORGA under the trade name GP4G®.

The agents stimulating the proliferation of fibroblasts that can be used in the composition according to the invention can for example be selected from plant proteins or polypeptides, extracted notably from soy (for example a soy extract marketed by the company LSN under the designation Eleseryl SH-VEG 8® or marketed by the company SILAB under the trade name Raffermine®); and plant hormones such as the gibberellins and the cytokinins.

The agents stimulating the proliferation of keratinocytes that can be used in the composition according to the invention notably comprise the retinoids such as retinol and its esters, including retinyl palmitate; adenosine; phloroglucinol; nut cake extracts marketed by the company GATTEFOSSE; and extracts of Solanum tuberosum marketed by the company SEDERMA.

The muscle relaxants or dermal relaxants that can be used in the compositions according to the invention comprise alverine and its salts, verapamil and its salts, dantrolene, manganese and its salts, in particular manganese gluconate, magnesium and its salts, diazepam, adenosine and derivatives thereof, the hexapeptide Argireline® marketed by the company LIPOTEC, certain carbonylated secondary and tertiary amines, adenosine, as well as sapogenins and natural extracts, in particular of Wild Yam.

“Lifting agent” means a compound capable of exerting a pulling action on the skin, which has the effect of temporarily smoothing the surface irregularities of the skin, such as wrinkles and lines.

Among the lifting agents that can be used in the composition according to the present invention, we may mention notably:

(1) synthetic polymers, such as polyurethane latexes or acrylic-silicone latexes,

(2) polymers of natural origin, notably (a) polyholosides, (b) latexes,

(3) proteins and hydrolyzates of plant proteins, in particular from maize, rye, wheat, buckwheat, sesame, spelt, pea, bean, lentil, soy and lupin,

(3) mixed silicates, notably phyllosilicates and in particular the laponites,

(4) wax microparticles,

(5) colloidal particles of inorganic filler having a number-average diameter between 0.1 and 100 nm, preferably between 3 and 30 nm.

The agents acting on the energy metabolism of the cells are, for example, and nonlimitatively, those acting on the synthesis of ATP, those which interfere with the respiratory pathway of the cell or on the energy stores. We may mention coenzyme Q10 (ubiquinone), cytochrome C, creatine, and phosphocreatine.

The soothing agents that can be used in the compositions according to the invention are notably extracts of plants of the genus Rosa, notably Rosa gallica, as described in EP906752, extracts of Iridaceae, notably of Iris pallida as described in EP765668, extracts of non-photosynthetic filamentous bacteria in particular of Vitreoscilla filiformis, as described for example in patent EP761204.

Other soothing agents that can be used in the composition according to the invention comprise: the pentacyclic triterpenes and plant extracts (e.g. Glycyrrhiza glabra) containing them such as β-glycyrrhetinic acid and its salts and/or derivatives (glycyrrhetinic acid monoglucuronide, stearyl glycyrrhetinate, 3-stearoyloxy glycyrrhetic acid), ursolic acid and its salts, oleanolic acid and its salts, betulinic acid and its salts, extracts of plants such as Paeonia suffruticosa and/or lactiflora, Laminaria saccharina, Boswellia serrata, Centipeda cunnighami, Helianthus annuus, Linum usitatissimum, Cola nitida, Epilobium angustifolium, Aloe vera, Bacopa monnieri, the salts of salicylic acid and in particular zinc salicylate, canola oil, bisabolol and chamomile extracts, allantoin, Sepivital EPC (phosphoric diester of vitamin E and C) from Seppic, unsaturated omega 3 oils such as musk rose oil, black currant oil, echium oil, or fish oil, plankton extracts, capryloyl glycine, Seppicalm VG (sodium palmitoylproline and Nymphea alba) from Seppic, tocotrienols, piperonal, a clove extract, phytosterols, salts of alkaline earth metals, notably of strontium, cortisone, hydrocortisone, indomethacin and betamethasone.

The compositions according to the invention can additionally contain at least one organic photoprotective agent and/or at least one inorganic photoprotective agent active in UVA and/or UVB (absorbers), water-soluble or fat-soluble or alternatively insoluble in the cosmetic solvents that are generally used.

The organic filters are notably selected from anthranilates; cinnamic derivatives; dibenzoylmethane derivatives; salicylic derivatives, camphor derivatives; triazine derivatives other than those of the invention such as those described in patent applications U.S. Pat. No. 4,367,390, EP863145, EP517104, EP570838, EP796851, EP775698, EP878469, EP933376, EP507691, EP507692, EP790243, EP944624; benzophenone derivatives; β,β-diphenylacrylate derivatives; benzotriazole derivatives; benzalmalonate derivatives; benzimidazole derivatives; imidazolines; derivatives of bis-benzoazolyl such as those described in patents EP669323 and U.S. Pat. No. 2,463,264; derivatives of p-aminobenzoic acid (PABA); derivatives of methylene bis-(hydroxyphenyl benzotriazole) such as those described in applications U.S. Pat. No. 5,237,071, U.S. Pat. No. 5,166,355, GB2303549, DE 197 26 184 and EP893119; the polymer filters and silicone filters such as those described notably in application WO-93/04665; the dimers derived from α-alkylstyrene such as those described in patent application DE19855649; the 4,4-diarylbutadienes as described in applications EP0967200, DE19746654, DE19755649, EP-A-1008586, EP1133980 and EP133981 and mixtures thereof.

As examples of organic filters, we may mention those designated below with their INCI name:

Derivatives of para-aminobenzoic acid:

### PABA,

### Ethyl PABA,

### Ethyl Dihydroxypropyl PABA,

Ethylhexyl Dimethyl PABA sold notably under the name

### “ESCALOL 507” by ISP,

### Glyceryl PABA,

PEG-25 PABA sold under the name “UVINUL P25” by BASE,

### Salicylic Derivatives:

Homosalate sold under the name “Eusolex HMS” by Rona/EM Industries, Ethylhexyl Salicylate sold under the name “NEO HELIOPAN OS” by HAARMANN & REIMER, Dipropyleneglycol Salicylate sold under the name “DIPSAL” by SCHER, TEA Salicylate, sold under the name “NEO HELIOPAN TS” by HAARMANN & REIMER,

### Derivatives of Dibenzoylmethane:

Butyl Methoxydibenzoylmethane sold notably under the trade name “PARSOL 1789” by HOFFMANN LA ROCHE, Isopropyl Dibenzoylmethane,

### Cinnamic Derivatives:

Ethylhexyl Methoxycinnamate sold notably under the trade name “PARSOL MCX” by HOFFMANN LA ROCHE,
Isopropyl Methoxy cinnamate,
Isoamyl Methoxy cinnamate sold under the trade name “NEO HELIOPAN E 1000” by HAARMANN & REIMER, DEA Methoxycinnamate,

### Diisopropyl Methylcinnamate,

### Glyceryl Ethylhexanoate Dimethoxycinnamate,

Derivatives of β,β′-diphenylacrylate:
Octocrylene sold notably under the trade name “UVINUL N539” by BASF,
Etocrylene, sold notably under the trade name “UVINUL N35” by BASF,

### Derivatives of Benzophenone:

Benzophenone-1 sold under the trade name “UVINUL 400” by BASF,
Benzophenone-2 sold under the trade name “UVINUL D50” by BASF,
Benzophenone-3 or Oxybenzone, sold under the trade name “UVINUL M40” by BASF,
Benzophenone-4 sold under the trade name “UVINUL MS40” by BASF,

### Benzophenone-5,

Benzophenone-6 sold under the trade name “Helisorb 11” by Norquay,
Benzophenone-8 sold under the trade name “Spectra-Sorb UV-24” by American Cyanamid,
Benzophenone-9 sold under the trade name UVINUL DS-49” by BASF,

### Benzophenone-12,

Diethylamino Hydroxybenzoyl Hexyl Benzoate sold under the trade name “UVINUL A PLUS” by BASF,

### Derivatives of Benzylidene Camphor:

3-Benzylidene camphor manufactured under the name “MEXORYL SD” by CHIMEX,
4-Methylbenzylidene camphor sold under the name “EUSOLEX 6300” by MERCK,
Benzylidene Camphor Sulfonic Acid manufactured under the name “MEXORYL SL” by CHIMEX,
Camphor Benzalkonium Methosulfate manufactured under the name “MEXORYL SO” by CHIMEX,
Terephthalylidene Dicamphor Sulfonic Acid manufactured under the name “MEXORYL SX” by CHIMEX,
Polyacrylamidomethyl Benzylidene Camphor manufactured under the name “MEXORYL SW” by CHIMEX,

### Derivatives of Phenyl Benzimidazole:

Phenylbenzimidazole Sulfonic Acid sold notably under the trade name “EUSOLEX 232” by MERCK,
Disodium Phenyl Dibenzimidazole Tetrasulfonate sold under the trade name “NEO HELIOPAN AP” by HAARMANN & REIMER,

### Derivatives of Triazine:

Bis-Ethylhexyloxyphenol Methoxyphenyl Triazine sold under the trade name “TINOSORB S” by CIBA GEIGY, Ethylhexyl triazone sold notably under the trade name “UVINUL T150” by BASF,
Diethylhexyl Butamido Triazone sold under the trade name “UVASORB HEB” by SIGMA 3V,

### Derivatives of Phenyl Benzotriazole:

Drometrizole Trisiloxane sold under the name “Silatrizole” by RHODIA CHIMIE,
Methylene bis-Benzotriazolyl Tetramethylbutylphenol, sold in solid form under the trade name “MIXXIM BB/100” by FAIRMOUNT CHEMICAL or in micronized form in aqueous dispersion under the trade name “TINOSORB M” by CIBA SPECIALTY CHEMICALS,

### Anthranilic Derivatives:

Menthyl anthranilate sold under the trade name “NEO HELIOPAN MA” by HAARMANN & REIMER,

### Derivatives of Imidazolines:

### Ethylhexyl Dimethoxybenzylidene Dioxoimidazoline Propionate,

### Derivatives of Benzalmalonate:

Polyorganosiloxanes with benzalmalonate function such as Polysilicone-15 sold under the trade name “PARSOL SLX” by HOFFMANN LA ROCHE,
Derivatives of 4,4-diarylbutadiene:
1,1-dicarboxy(2,2′-dimethyl-propyl)-4,4-diphenylbutadiene,
   Derivatives of benzoxazole:
   2,4-bis-[5-(1-dimethylpropyl)benzoxazol-2-yl-(4-phenyl)-imino]-6-(2-ethylhexyl)-imino-1,3,5-triazine sold under the name Uvasorb K2A by Sigma 3V
   and mixtures thereof.

The preferred supplementary organic UV filters are selected from:

### Ethylhexyl Salicylate,

### Homosalate,

### Ethylhexyl Methoxycinnamate,

### Butyl Methoxydibenzoylmethane,

### Octocrylene,

### Phenylbenzimidazole Sulfonic Acid,

### Disodium Phenyl Dibenzimidazole Tetrasulfonate,

### Benzophenone-3,

### Benzophenone-4,

### Benzophenone-5,

N-Hexyl 2-(4-diethylamino-2-hydroxybenzoyl)-benzoate,
4-Methylbenzylidene camphor,

### Terephthalylidene Dicamphor Sulfonic Acid,

### Bis-Ethylhexyloxyphenol Methoxyphenyl Triazine

Ethylhexyl triazone,

### Diethylhexyl Butamido Triazone,

Methylene bis-Benzotriazolyl Tetramethylbutylphenol,

### Drometrizole Trisiloxane,

### Polysilicone-15,

1,1-Dicarboxy(2,2′-dimethyl-propyl)-4,4-diphenylbutadiene,
2,4-bis-[5-(1-dimethylpropyl)benzoxazol-2-yl-(4-phenyl)-imino]-6-(2-ethylhexyl)-imino-1,3,5-triazine
and mixtures thereof.

The supplementary inorganic photoprotective agents are selected from pigments and more preferably from nanopigments (average size of the primary particles: generally between 5 nm and 100 nm, preferably between 10 nm and 50 nm) of treated or untreated metal oxides, for example nanopigments of titanium dioxide (amorphous or crystalline in the rutile and/or anatase form), iron oxide, zinc oxide, zirconium oxide or cerium oxide.

The treated nanopigments are pigments that have undergone one or more surface treatments of a chemical, electronic, mechanochemical and/or mechanical nature with compounds such as are described for example in Cosmetics & Toiletries, February 1990, Vol. 105, p 53-64, such as amino acids, beeswax, fatty acids, fatty alcohols, anionic surfactants, lecithins; sodium, potassium, zinc, iron, or aluminum salts of fatty acids; metal alkoxides (of titanium or aluminum), polyethylene, silicones, proteins (collagen, elastin), alkanolamines, silicon oxides, metal oxides, sodium hexametaphosphate, alumina or glycerol.

The nanopigments can be added to the compositions according to the invention as they are or in the form of pigment paste, i.e. mixed with a dispersant, as described for example in document GB-A-2206339.

The additional photoprotective agents are generally present in the compositions according to the invention in proportions ranging from 0.01 to 20 wt. % relative to the total weight of the composition, and preferably ranging from 0.1 to 10 wt. % relative to the total weight of the composition.

The invention also relates to a cosmetic method for combating age-related changes of the skin, in which at least one PBR antagonist or a composition containing it, as defined in the foregoing, is applied to the skin or its appendages. The method will be particularly suitable for preventing or reducing the signs of intrinsic ageing. Application can be daily or several times a day; it can be of long duration and can notably last for 2 to 8 weeks or more, in repeated or continuous treatments. This method is suitable for combating wrinkles or lines, notably laugh-lines, for combating thinning of the epidermis and the loss of splendor of the skin, and/or for combating dehydration of the skin. It can be applied to all areas of the body that are affected, notably the face, the neck and shoulders, the arms and/or the legs. This treatment can be used advantageously for women or for men aged 25 years or over, in particular over 40 years, for whom we observe a physiological increase in the changes described in the foregoing, which can be limited, reduced or delayed by the application of PBR antagonist.

The examples given below are intended to illustrate the invention without limiting it.

### EXAMPLE 1

### Differentiation of Keratinocytes—Determination of TGK1 Activity

### Protocol:

Normal human epidermal keratinocytes are cultivated at 37° C. in keratinocyte-SFM medium (Gibco BRL 3701022) supplemented with EGF (epidermal growth factor) and pituitary extract, with or without calcium supplement (1.5 mM). After culture for 24 hours, the medium is replaced with medium containing the actives to be tested or reference substances. Culture is continued for 96 hours. The transglutaminase K1 (TGK1) activity is evaluated by radioassay, by measuring the covalent addition of tritiated putrescine to casein (acceptor protein) for 1 h 30 min at 37° C. The casein is precipitated with TCA (trichloroacetic acid) on a dry filter and then counted in liquid scintillation. The proteins in each sample are determined by means of a kit (BioRad 500-0116). Statistical analysis of the data is performed by analysis of variance (ANOVA) using Dunnett's multiple comparison test.

The molecule PK11195 (or 1-(2-chlorophenyl)-N-(1-methyl-propyl)-3-isoquinoline carboxamide), a specific inhibitor of the peripheral benzodiazepine receptor, was tested at respective concentrations of 10⁻⁴ M and 10⁻⁶ M. The active was added to the keratinocyte culture medium, containing calcium or not containing calcium. The effect of a medium alone containing 1.5 mM of calcium (pro-differentiation agent) was used as a positive control of differentiation. Conversely, retinol at a concentration of 10⁻⁶M was used as anti-differentiation reference.

### Results:

Medium without calcium

| | |
|---|---|
| 10⁻⁴ M | 10⁻⁶ M |

| | TGK1 | Cellular | TGK1 | Cellular |
|---|---|---|---|---|
| | activity | viability | activity | viability |
| Control | 100% | 100% | 100% | 100% |
| Control + | 281% | 82% | 244% | 82% |
| calcium | | | | |
| PK11195 | 24% | 0 | 82%* | 107% |

Medium with calcium

| | |
|---|---|
| 10⁻⁴ M | 10⁻⁶ M |

| | TGK1 | Cellular | TGK1 | Cellular |
|---|---|---|---|---|
| | activity | viability | activity | viability |
| Control | 100% | 100% | 100% | 100% |
| Retinol | ND | ND | 56% | 103% |
| PK11195 | 21% | 30% | 81%* | 103% |

The results of this study show that the molecule PK11195 at a concentration of 10⁻⁶ M exerts pro-differentiation effects, with or without calcium in the culture medium. The average decrease in TGK1 activity is 20% and is statistically significant (*p<0.05).

The reference substances produced the expected effects, i.e. an increase in TGK1 activity in the case of the medium with calcium (+281 and 244% on the 2 culture plates) and a decrease in TGK1 activity in the case of a medium with retinol 10⁻⁶ M (−44%).

### EXAMPLE 2

### Synthesis of Total Epidermal Glycosaminoglycans

### Protocol:

Normal human epidermal keratinocytes are cultivated in SFM complete medium for 24 hours. The medium is then removed, and replaced with SFM medium without supplements containing the product PK11195 or the pharmacological reference. It is incubated for 72 h at 37° C. Tritiated glucosamine (D-[6-³H]-glucosamine, Amersham TRK398 (1.3 Tbq/mmol, 35 Ci/mmol) is added for the last 24 hours of incubation. The glycosaminoglycans are extracted from the culture medium and from the deposited matrix, with a chaotropic buffer, then purified by ion-exchange chromatography. The radioactivity incorporated in the highly cationic molecules (mostly GAGs) is counted by liquid scintillation. The results are expressed as percentage change in synthesis of the GAGs relative to the control.

### Results:

| Incorporation of ³H-glucosamine | | | | |
|---|---|---|---|---|
| | | | Percentage/ | |
| | Concentration | CPM | control | p |

| Control | — | | 10624 | — | — |
|---|---|---|---|---|---|
| CaCl₂ | 1.5 | mM | 16791 | 158% | <0.01 |
| PK11195 | 10⁻⁵ | M | 12477 | 117% | <0.05 |

The product PK11195, a peripheral benzodiazepine receptor antagonist, significantly increases the incorporation of tritiated glucosamine. This signifies that this molecule stimulates the synthesis of glycosaminoglycans by normal human keratinocytes in culture.

### EXAMPLE 3

### Effect on the Contraction of Collagen Lattices by Human Dermal Fibroblasts in Culture

The principle of this test comprised investigating the relaxant effect of a peripheral benzodiazepine receptor antagonist on a dermis equivalent model comprising a collagen matrix seeded with normal human fibroblasts.

These conditions are intended to simulate in vitro the contractile phenomena of the skin which occur during facial expressions. In fact, in these conditions the cells spontaneously express pulling forces which cause shrinkage of the collagen gel. This leads to a decrease in total surface area of the dermis equivalent over time. By measuring this surface area, we are able to evaluate the relaxation effects of the substances previously brought into contact with the dermis equivalent.

### Protocol:

The lattices are prepared in 24-well plates (TO), in a complete fibroblast medium (DMEM, L-glutamine 2 mM, penicillin/streptomycin 50 IU/ml/50 μg/ml; without serum) containing rat tail collagen (Institut J. Boy; 1.3 mg/ml final) and 10⁶ cells/ml (normal human dermal fibroblasts used in the 9th passage). The volume of suspension is 400 μl per well.

After one hour at 37° C. (collagen gelation time), the lattices are detached from the support and 1 ml of culture medium, containing bradykinin (0.1 μM final) and product PK11195 at a concentration of 10⁻⁶ M, was added per culture well. The plates were incubated at 37° C. and 5% CO₂ and the size of the lattices was measured after 5 hours. The surface of the lattices was analyzed using Lucia software after image processing.

### Results:

| | Contraction of the collagen lattices in the | | | |
|---|---|---|---|---|
| | presence of bradykinin 0.1 μM | | | |
| | | Lattice mean | | |
| | | surface area | Percentage/ | |
| | Concentration | (mm²) | control | p |
| Control | — | 89.33 | — | — |
| PK11195 | 10⁻⁶ M | 94.02 | 113% | <0.01 |

The presence of bradykinin causes rapid contraction of the collagen lattice, simulating the physiological conditions of pulling of the normal dermis. The product PK11195, a peripheral benzodiazepine receptor antagonist, significantly increases the surface area of the collagen lattices after culture for 6 hours in the presence of bradykinin. This means that PK11195 is capable of inducing relaxation of the lattice and therefore exerts a dermorelaxant effect on this lattice.

### EXAMPLE 4

### Anti-Age Composition

| Oil-in-water emulsion | |
|---|---|
| Compound 1(16) | 0.30% |
| Lyophilized extract of rosemary | 0.20% |
| Glycerol stearate | 2.00% |
| Polysorbate 60 | 1.00% |
| Stearic acid | 1.40% |
| Triethanolamine | 0.70% |
| Carbomer | 0.40% |
| Olive oil | 12.00% |
| Liquid fraction of shea butter | 12.00% |
| Octyldodecanol | 6.00% |
| Isononyl isononanoate | 10.00% |
| Antioxidant | 0.05% |
| Perfume | 0.50% |
| Preservative | 0.30% |
| Water qsf | 100% |

This composition can be applied morning and evening on all of the face and neck.

| Night cream | |
|---|---|
| Compound 2 (105) | 0.50% |
| Retinol | 0.10% |
| Glycerol | 3.00% |
| Xanthan gum | 0.10% |
| Oxyethylenated sorbitan stearate | 0.90% |
| PEG-100 stearate and glyceryl stearate | 2.10% |
| Cetyl alcohol | 2.60% |
| Isononyl isononanoate | 11.00% |
| Octyldodecanol | 15.00% |
| Butylhydroxytoluene | 0.10% |
| Octocrylene | 2.00% |
| Triethanolamine | 0.30% |
| Tocopherol acetate | 1.00% |
| Preservatives | 0.60% |
| Water qsf | 100% |

This cream is applied every evening on all of the face: and neck to combat wrinkles and lines and loss of splendor of the skin.

1. A method of reducing or preventing at least one sign of skin ageing in a human in need thereof, comprising applying a composition comprising at least one antagonist of the peripheral benzodiazepine receptors in a physiologically acceptable medium to the skin of the human in need thereof in an amount sufficient to reduce or prevent the at least one sign of skin ageing.

2. The method of claim 1, wherein the skin ageing is chronological ageing.

3. The method of claim 1, wherein the reducing or preventing at least one sign of skin ageing comprises reducing the differentiation of keratinocytes, promoting the proliferation of keratinocytes, or reducing the differentiation of keratinocytes and promoting the proliferation of keratinocytes.

4. The method of claim 1, wherein the reducing or preventing at least one sign of skin ageing comprises promotion of the synthesis of epidermal macromolecules.

5. The method of claim 1, wherein the reducing or preventing at least one sign of skin ageing comprises relaxing at least one tissue selected from the group consisting of cutaneous tissue and subcutaneous tissue.

6. The method of claim 1, wherein the reducing or preventing at least one sign of skin ageing comprises combating thinning of the skin associated with age.

7. The method of claim 1, wherein the reducing or preventing at least one sign of skin ageing comprises combating at least one sign selected from the group consisting of a wrinkle and a line.

8. The method of claim 1, wherein the reducing or preventing at least one sign of skin ageing comprises reducing or preventing at least one laugh-line.

9. The method of claim 1, wherein the reducing or preventing at least one sign of skin ageing comprises combating at least one sign selected from the group consisting of flabby skin and withered skin.

10. The method of claim 1, wherein the reducing or preventing at least one sign of skin ageing comprises combating at least on sign selected from the group consisting of lack of skin tone and lack of skin elasticity.

11. The method of claim 1, wherein the reducing or preventing at least one sign of skin ageing comprises combating dehydration of the skin.

12. The method of claim 1, wherein at least one antagonist of the peripheral benzodiazepine receptors is present in the composition at a concentration from 0.001% to 20% of the total weight of the composition.

13. (canceled)

14. The method of claim 1, wherein the applying is a topical, external applying.

15. The method of claim 1, wherein the applying is conducted via an oral route.

16. The method of claim 1, wherein the composition further comprises at least one compound selected from the group consisting of at least one anti-UVA filter, at least one anti-UV B filter, at least one hydrating agent, at least one depigmenting agent, at least one antiglycation agent, at least one inhibitor of NO synthase, at least one dermal macromolecule stimulation agent, at least one epidermal macromolecule stimulation agent, at least one epidermal macromolecule degradation preventative, at least one dermal macromolecule degradation preventative, at least one agent that stimulates the proliferation of fibroblasts, at least one agent that stimulates the proliferation of keratinocytes, at least one muscle relaxant, at least one dermal relaxant, at least one lifting agent, at least one antipollution agent, at least one antiradical agent, at least one soothing agent, and at least one substance acting on the energy metabolism of a cell.

17. The method of claim 1 wherein the at least one antagonist comprises 1-(2-chlorophenyl)-N-(1-methyl-propyl)-3-isoquinoline carboxamide.

18. A method of reducing or preventing at least one sign of mucosae ageing in a human in need thereof, comprising applying a composition comprising at least one antagonist of the peripheral benzodiazepine receptors in a physiologically acceptable medium to a mucosae of the human in need thereof in an amount sufficient to reduce or prevent the at least one sign of mucosae ageing.

19. The method of claim 1, wherein the at least one sign of skin ageing is at least one sign selected from the group consisting of a wrinkle, a line, thinning of the epidermis, loss of skin splendor, and dehydration of the skin.

20. The method of claim 1, wherein the method is a method of reducing.

21. The method of claim 1, wherein the method is a method of preventing.
